(19) United States
(12) Patent Application Publication
(10) Pub. No.: US 2019/0060384 A1
(43) Pub. Date: Feb. 28, 2019

(54) COMPOSITION FOR TREATMENT OR PREVENTION OF LUNG CANCER AND A METHOD THEREFORE

(71) Applicant: COMPREHENSIVE AND INTEGRATIVE MEDICINE INSTITUTE, Daegu (KR)

(72) Inventors: Sang Hoon Joo, Daegu (KR); Young Joon Lee, Daegu (KR); Sae-Kwang Ku, Daegu (KR); Joon-Seok Byun, Daegu (KR); Ki Cheul Sohn, Daegu (KR)

(73) Assignee: COMPREHENSIVE AND INTEGRATIVE MEDICINE INSTITUTE, Daegu (KR)

(21) Appl. No.: 16/105,511

(22) Filed: Aug. 20, 2018

(30) Foreign Application Priority Data
Aug. 23, 2017 (KR) 10-2017-0106937

(51) Int. Cl. A61K 36/355; A61K 31/5377; A61P 35/00

(52) U.S. Cl. CPC A61K 36/355; A61K 9/0053; A61P 35/00; A61K 31/5377

(57) ABSTRACT

The present invention relates to a composition for the treatment or prevention of lung cancer, including gefitinib and a Lonicera japonica extract, and more particularly, the present invention has an advantage in that it is possible to remarkably increase the therapeutic effects of gefitinib on lung cancer and simultaneously prevent and reduce side effects caused by various treatments of lung cancer, and the like by administering gefitinib to an individual and administering a Lonicera japonica extract to the individual.

### CROSS-REFERENCE TO RELATED APPLICATION

This application claims priority of Korean Patent Application No. 10-2017-0106937, filed Aug. 23, 2017, the contents of which is incorporated herein by reference in its entirety.

### FIELD OF THE INVENTION

The present invention provides a composition for treatment or prevention of lung cancer, including gefitinib and a Lonicera japonica extract.

### BACKGROUND OF THE INVENTION

As an anticancer therapy for lung cancer, various methods such as treatment using surgery, radiation therapy, anticancer chemotherapy, a target therapeutic agent, and bronchoscopy have been attempted for the treatment of non-small cell lung cancer according to the disease stage. In general, it is known that 30 to 50% and 20 to 35% of lung cancer patients respond to radiation therapy and anticancer chemotherapy, respectively. Examples of a target therapeutic agent that has recently drawn attention include bevacizumab, gefitinib, intedanib, and the like.

Among the anticancer agents for the treatment of lung cancer, gefitinib is the first developed drug which selectively suppresses the tyrosine kinase activity of the epidermal growth factor receptor (EGFR), such EGFR is usually overexpressed in a solid tumor such as lung cancer or breast cancer, and it is known that the overexpression of EGFR plays an important role in the growth of cancer cells through the suppression of cell replication and apoptosis, the angiogenesis of cancer cells, and the like. Gefitinib blocks the autophsphorylation of EGFR by selectively inhibiting the activity of tyrosine kinase of such EGFR, and as a result, suppresses the proliferation of cancer cells by completely blocking the signal transduction from EGFR. Unlike other existing anticancer agents that have effects on small cell lung cancer, but have little therapeutic effects on non-small cell lung cancer, gefitinib also exhibits therapeutic effects on non-small cell lung cancer, and for this reason, gefitinib has drawn attention as the only alternative drug for non-small cell lung cancer which has failed in existing anticancer treatment.

Although gefitinib is a therapeutic agent having very low toxicity as compared to the existing cytotoxic anticancer agents, it also exhibits various unintended side effects such as a skin rash, diarrhea, nausea, vomiting, anorexia, gastritis, dehydration, hepatotoxicity, a feeling of helplessness, conjunctivitis, blepharitis, interstitial lung disease, corneal erosion, and milphosis.

Meanwhile, Lonicera japonica is a medicinal herb, in which flower buds of Lonicera japonica Thunb. belonging to Caprifoliaceae or flowers thereof just beginning to bloom are dried, has been used for various traditional Korean medicinal prescriptions [Ku et al., 2009], and is known to have excellent body temperature regulation, anti-pyretic, detoxification, and anti-inflammation effects.

Thus, in the present invention, while a method capable of reducing toxicity or side effects caused by the use of gefitinib had been studied, it was confirmed that when a Lonicera japonica extract was treated, side effects could be reduced while increasing therapeutic effects of gefitinib, thereby completing the present invention.

### SUMMARY OF THE INVENTION

Thus, the present invention has been contrived to address the needs in the related art as described above, and an object thereof is to provide a composition which enhances the treatment, alleviation, and prevention effects of lung cancer symptoms and reduces side effects caused by the treatment of lung cancer by using a Lonicera japonica extract.

However, a technical problem to be achieved by the present invention is not limited to the aforementioned problem, and the other problems that are not mentioned may be clearly understood by a person skilled in the art from the following description.

In order to achieve the object, the present invention provides a composition for treatment or prevention of lung cancer, including gefitinib and a Lonicera japonica extract.

As another exemplary embodiment of the present invention, the composition is characterized by reducing side effects caused by the treatment of lung cancer.

As still another exemplary embodiment of the present invention, the gefitinib and the Lonicera japonica extract are characterized by being formulated by being mixed in advance, or being formulated separately.

As yet another exemplary embodiment of the present invention, the gefitinib and the Lonicera japonica extract are characterized by being parenterally, orally, locoregionally, or percutaneously administered.

As still yet another exemplary embodiment of the present invention, the administration of the Lonicera japonica extract is characterized by beginning between 5 minutes and 4 hours after the administration of the gefitinib.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a diagram illustrating an experimental design of Example 2;

FIG. 2 is a graph illustrating a result of observing effects of LF on the cell viability of NCI-H520 in Example 2;

FIG. 3 is a graph illustrating a result of observing effects of gefitinib on the cell viability of NCI-H520 in Example 2;

FIG. 4 is a graph illustrating a result of identifying a change in body weight according to each group in Example 2;

FIG. 5 is a set of photographs showing the size of a tumor according to each group in Example 2;

FIG. 6 is a graph illustrating the results of observing the size of a tumor according to each group in Example 2;

FIG. 7 is a graph illustrating a change in serum IL-6 and IFN-γ contents according to each group in Example 2;

FIG. 8 is a graph illustrating a change in NK cell activity according to each group in Example 2;

FIG. 9 is a set of photographs illustrating results of observing histopathological changes of a tumor mass in Example 2;

FIG. 10 is a set of photographs illustrating results of observing a change in the number of caspase-3 and PARP in a tumor mass in Example 2;

FIG. 11 is a set of photographs illustrating results of observing a change in the number of NT and 4-HNE in a tumor mass in Example 2;

FIG. 12 is a set of photographs view illustrating results of observing a change in the number of Ki-67 and CD31 in a tumor mass in Example 2;

FIG. 13 is a set of photographs illustrating results of observing a change in the number of COX-2, iNOS, and TNF-α in a tumor mass in Example 2;

FIG. 14 is a set of photographs illustrating results of observing histopathological changes of a spleen in Example 2;

FIG. 15 is a set of photographs illustrating results of identifying histopathological changes of a submandibular lymph node in Example 2;

FIG. 16 is a set of photographs illustrating results of identifying histopathological changes of a periovarian fat pad in Example 2;

FIG. 17 is a view illustrating an experimental design of Example 3;

FIG. 18 is a graph illustrating a result of identifying a change in body weight according to each group in Example 3;

FIG. 19 is a set of photographs illustrating results of observing histopathological changes of a spleen in Example 3;

FIG. 20 is a set of photographs illustrating results of identifying histopathological changes of a submandibular lymph node in Example 2;

FIG. 21 is a set of photographs illustrating results of identifying histopathological changes of a liver in Example 2;

FIG. 22 is a set of photographs illustrating results of observing histopathological changes of a lung in Example 3; and

FIG. 23 is a set of photographs illustrating results of identifying histopathological changes of the cardiac stomach in Example 3.

### DETAILED DESCRIPTION OF THE INVENTION

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

The present inventors have concentrated on natural materials in order to develop a composition capable of further increasing an effect of decreasing the volume of a tumor while being capable of reducing various side effects occurring in the case of administering a representative EGFR inhibitor, gefitinib, which is an oral anticancer agent used for the treatment of lung cancer, and confirmed that among the natural materials, a Lonicera japonica extract has an excellent effect of enhancing the treatment of lung cancer and an effect of reducing side effects, thereby completing the present invention.

Therefore, an object of the present invention is to provide a pharmaceutical composition for treatment or prevention of lung cancer, including gefitinib and a Lonicera japonica extract.

That is, the composition is characterized by reducing side effects caused by the treatment with gefitinib, which is an anticancer chemotherapeutic.

As an exemplary embodiment of the present invention, the gefitinib and the Lonicera japonica extract may be formulated by being mixed in advance, or may be formulated separately.

For the administration period of the Lonicera japonica extract, the Lonicera japonica extract may be administered within 5 minutes to 4 hours, may be administered preferably within 30 minutes to 3 hours, and may be administered most preferably within 30 minutes to 2 hours and 30 minutes, after the gefitinib is administered, and the Lonicera japonica extract may be administered 2 hours after gefitinib is administered, but the administration period is not limited thereto.

Gefitinib, a representative EGFR inhibitor, used in the present invention is an oral anticancer agent which is frequently used as a therapeutic agent against various malignant tumors including breast cancer and lung cancer, is usually known to suppress the EGFR tyrosine kinase domain and known to have very low toxicity as a target-oriented anticancer agent as compared to the existing cytotoxic anticancer agents, but causes not only various unintended side effects such as a skin rash, diarrhea, nausea, vomiting, anorexia, gastritis, dehydration, paronychia, hepatotoxicity, a feeling of helplessness, conjunctivitis, blepharitis, interstitial lung disease, corneal erosion, and milphosis, but also hypersensitive responses to gefitinib itself or constituent ingredients thereof, and an increase in lipid peroxidation caused by metabolites formed in the liver and the hepatotoxicity caused by damage to the antioxidant defense system accompanied by the increase in lipid peroxidation have been problematic, and recently, due to the emergence of resistant malignant tumor cells caused by the mutation of EGFR, and the like, efforts to solve the toxicity and resistance problems of gefitinib caused by combination administration of natural materials and drugs including various antioxidants have been attempted.

The gefitinib and the Lonicera japonica extract may be parenterally, orally, locoregionally, or percutaneously administered. It is preferred that the Lonicera japonica extract is orally administered, but the administration route thereof may be appropriately selected by a person with ordinary skill in the art, depending on the condition and body weight of a patient, the degree of a disease, the duration, and the like.

In the present invention, “an individual” refers to a subject in need of treatment of a disease, and more specifically, refers to a mammal such as a human or a non-human primate, a mouse, a rat, a dog, a cat, a horse, and a cow.

Further, the present invention can provide a method for treating lung cancer, including a Lonicera japonica extract.

The pharmaceutical composition of the present invention may include a pharmaceutically acceptable carrier. The pharmaceutically acceptable carrier may include physiological saline, polyethylene glycol, ethanol, vegetable oil, isopropyl myristate, and the like, and is not limited thereto.

In an exemplary embodiment of the present invention, a preferred dosage of the pharmaceutical composition varies depending on the condition and body weight of a patient, the degree of a disease, the form of drug, the administration route, and the duration, but may be appropriately selected by the person skilled in the art. However, the pharmaceutical composition is administered preferably at 0.001 to 300 mg/kg of body weight daily, and more preferably at 0.01 to 200 mg/kg of body weight daily.

The pharmaceutical composition of the present invention may be administered to a mammal such as a rat, a mouse, livestock, and a human via various routes. The administration method is not limited, and the pharmaceutical composition of the present invention may be administered by oral, rectal, or intravenous, intramuscular, hypodermic, intrauterine, or intracerebroventricular injections.

A composition for treatment or prevention of lung cancer, including the Lonicera japonica extract of the present invention has effects capable of increasing an effect of treating lung cancer and capable of simultaneously reducing various side effects caused when gefitinib is conventionally administered alone.

### EXAMPLES

Hereinafter, preferred Examples for helping the understanding of the present invention will be suggested. However, the following Examples are provided only to more easily understand the present invention, and the contents of the present invention are not limited by the following Examples.

### Examples

### Example 1

### Development of Complex Preparation of Therapeutic Agent for Lung Cancer Gefitinib (Iressa™) and Hot-Water Extract Lyophilisate of Lonicera japonica (LF): Effects of LF Complex Composition on Anticancer Effects of Gefitinib in NCI-H520 Non-Small Cell Lung Cancer Cell Xenograft Nude Mice—Single Oral Administration

In the present Example 1, in order to find a combination administration of a new Oriental and Western medical therapeutic agent for treatment of lung cancer, after Lonicera flos and gefitinib were administered singly in combination with each other within 5 minutes, the non-compartmental pharmacokinetics data of gefitinib (Cmax, Tmax, AUC, t1/2, and MRT) were calculated and each analyzed in comparison with those of a single administration group of gefitinib, and after Lonicera flos and gefitinib were administered singly and repeatedly at a predetermined interval, effects of gefitinib on the pharmacokinetics were also observed.

As a Lonicera japonica extract reagent, a Lonicera flos powder (College of Pharmacy at Dongguk University, Goyang, Korea) was purchased and used, and gefitinib (Santa Cruz, Tex., USA), DMSO, formic acid (Sigma-Aldrich, Missouri, USA), and saline (JW Pharmaceutical, Seoul, Korea) were used.

1.1. Preparation of Experimental Animals and Separation of Groups

In the present study, male Sprague-Dawley rats (5 weeks, 130 to 170 g) (OrientBio, Seongnam, Korea) were used. In a plastic cage, the rats were bred with free access to water and a rat diet, and used in a test after a 7-day acclimatization period. The test was carried out in a state where a light/dark cycle of 12 hours was maintained at a temperature of 23±2° C. and a humidity of 50±10%.

Dose Setting of Lonicera flos and Production of Administration Solution

A dose of Lonicera flos was determined in consideration of a usual dosage in a human body and the stability of a suspension. The dose of Lonicera flos in the human body was each 2 or 3 g/day, and when the dose is converted into a dose per unit body weight in the human body, the dose was each calculated into 300 and 500 mg/kg, and administered to the rats. The Lonicera flos powder was suspended in the saline and administered.

1.3. Administration Method

(1) Repeated Administration of Lonicera flos

The Lonicera flos dispersed in saline was each prepared at a dose of 300 and 500 mg/kg, and repeatedly administered orally at a volume of 5 mL/kg to an experimental group for 15 days. In order to exclude the effects caused by a base material, the same volume of saline was also repeatedly administered orally to a control for 15 days. For the administration, the oral administration to the rats was performed by connecting an oral zonde needle. A predetermined amount of feed was supplied for 15 days after the beginning of administration, the fasting state was maintained by stopping the supply of the feed 14 hours before the final administration (day 15), and the feed was supplied again after the time point of collecting blood 4 hours after administration. Within the experimental period, the drinking water was unlimitedly supplied.

(2) Administration of Gefitinib

Gefitinib was orally administered within 5 minutes after administering Lonicera flos on day 15 after the beginning of administering Lonicera flos. The dose of gefitinib was 10 mg/kg, and gefitinib was administered at a volume of 2 mL/kg by being dissolved in 10% DMSO, 40% PEG400, 49% saline, and 1% 0.1 N HCl. For the administration, the oral administration to the rats was performed by connecting an oral zonde needle. The fasting state was maintained at the time of administration by stopping the supply of the feed for 14 hours or more prior to the administration, and the feed was supplied again after the time point of collecting blood at the 3rd hour. Within the experimental period, the drinking water was unlimitedly supplied.

(3) Collection and Analysis of Sample

For a blood sample, blood was collected at 30 minutes before administration of gefitinib and 15 and 30 minutes and 1, 3, 7, 10, and 24 hours after administration of gefitinib. At each blood collection time point, 0.3 mL of blood was collected from the jugular vein of the rats. Thereafter, blood was centrifuged at 13,500 rpm for 5 minutes, and then blood plasma was taken and stored at −20° C. until analysis. After 50 μL of blood plasma was taken from the frozen stored tube, 50 μL of elotinib, which is an internal standard, and 150 μL of 0.1% formic acid in acetonitrile were added thereto. Thereafter, the mixture was vortexed for 10 minutes and centrifuged at 13,500 rpm for 10 minutes, and then gefitinib, which is an administered drug, was quantified by using the LC/MS/MS simultaneous analysis method. The animal test contents are summarized in the following Table 1. The pharmacokinetic parameters of gefitinib were obtained by using a Winonline program.

TABLE 1

| Administration | | |
|---|---|---|
| group (n = | | |
| 10) | Lonicera flos | Administration of gefitinib |
| Control | N/A | Oral, Administered singly |
| Group of | Once daily, orally | Oral, Administered singly |
| Lonicera flos | administered 15 times | |
| | in total | |

1.3. Result Confirmation

In order to evaluate effects of combination administration of gefitinib after repeated administration of Lonicera flos on the pharmacokinetic behavior of gefitinib, the administration group was divided into a control to which gefitinib was administered within 5 minutes after saline was orally administered and a test group to which gefitinib was administered within 5 minutes after administration on day 15 after Lonicera flos was repeatedly administered orally at 300 mg/kg for 15 days, and then the in vivo behavior of gefitinib were evaluated.

The analysis equipment used is shown in the following Table 2.

TABLE 2

| | |
|---|---|
| LC/MS/ | AB SCIEX API 4000 Q TRAP coupled with Agilent 1100 |
| MS system | HPLC system (AB SCIEX, Framingham, MA, USA; |
| | Agilent Technologies, Santa Clara, CA, USA) |
| Integrator | Analyst 1.5.2. program (Agilent Technologies, SantaClara, |
| | CA, USA) |
| Guard | Narrow-Bore Guard Column 2.112.5 mm 5-Micron |
| column | (Agilent Technologies, Santa Clara, CA, USA) |
| Centrifuge | Centrifuge Smart-15 (HANIL, Daejeon, Korea) |
| Balance | Sartorius Weighing Technology GmBH CPA124S |
| | 27708592 (Sartorius, Goettingen, Germany) |

Observation of Serum Gefitinib Concentration

For gefitinib obtained from the blood collected from each group, the average plasma concentration-time data and the individual plasma concentration-time data were obtained as in the following Tables 3 and 4. The average pharmacokinetic parameters and the individual pharmacokinetic parameters of each drug calculated from the plasma concentration-time curve are shown as in Table 5.

The orally administered gefitinib exhibited the maximum plasma concentration at about 4 hours after administration. The pharmacokinetic parameter aspects including the last area under the curve (AUClast) and the maximum plasma concentration (Cmax) of each of the Lonicera flos and the Korean angelica group exhibited an aspect in which the pharmacokinetic parameters were decreased on the whole as compared to those of the control. However, when the data was statistically processed by one-way ANOVA, the pharmacokinetic parameters of the control and the Lonicera flos group or the Korean angelica group had no significant difference. Therefore, it can be concluded that the repeated oral administration of Lonicera flos or Korean angelica group does not have any significant effects on the pharmacokinetic behavior of gefitinib.

TABLE 3

| Time | Plasma concentration (μg/mL) | | | | | | | | | | | |
|---|---|---|---|---|---|---|---|---|---|---|---|---|
| (h) | #1 | #2 | #3 | #4 | #5 | #6 | #7 | #8 | #9 | #10 | mean | S.D. |
| 0.25 | 0.404 | 0.211 | 0.319 | 0.594 | 0.651 | 0.311 | 0.610 | 0.674 | 0.234 | 0.509 | 0.442 | 0.170 |
| 0.50 | 0.750 | 0.507 | 0.675 | 0.876 | 0.614 | 0.361 | 0.768 | 0.997 | 0.425 | 1.060 | 0.703 | 0.234 |
| 1 | 0.863 | 0.537 | 0.861 | 0.787 | 1.230 | 0.692 | 0.808 | 1.070 | 0.463 | 0.748 | 0.805 | 0.227 |
| 3 | 0.951 | 0.501 | 0.896 | 0.980 | 1.080 | 0.464 | 0.633 | 0.913 | 0.437 | 0.774 | 0.762 | 0.237 |
| 7 | 1.060 | 0.686 | 0.895 | 1.190 | 0.806 | 0.429 | 0.785 | 0.910 | 0.520 | 0.958 | 0.824 | 0.233 |
| 10 | 0.630 | 0.392 | 0.543 | 0.055 | 0.482 | 0.202 | 0.552 | 0.629 | 0.231 | 0.475 | 0.439 | 0.227 |
| 24 | 0.104 | 0.060 | 0.034 | 0.027 | 0.076 | 0.068 | 0.055 | 0.124 | 0.043 | 0.107 | 0.070 | 0.033 |

TABLE 4

| Time | Plasma concentration (μg/mL) | | | | | | | | | | |
|---|---|---|---|---|---|---|---|---|---|---|---|
| (h) | #1 | #2 | #3 | #4 | #5 | #6 | #7 | #8 | #9 | mean | S.D. |
| 0.25 | 0.243 | 0.260 | 0.084 | 0.467 | 0.269 | 0.167 | 0.184 | 0.514 | 0.262 | 0.274 | 0.138 |
| 0.50 | 0.521 | 0.366 | 0.141 | 0.846 | 0.353 | 0.574 | 0.680 | 0.984 | 0.677 | 0.571 | 0.261 |
| 1 | 0.648 | 0.572 | 0.093 | 0.830 | 0.579 | 0.621 | 0.608 | 0.896 | 0.961 | 0.645 | 0.253 |
| 3 | 0.657 | 0.239 | 0.259 | 0.681 | 0.484 | 0.620 | 0.461 | 0.679 | 0.701 | 0.531 | 0.181 |
| 7 | 0.833 | 0.354 | 0.496 | 0.894 | 0.229 | 0.470 | 0.420 | 0.719 | 0.798 | 0.579 | 0.237 |
| 10 | 0.610 | 0.185 | 0.415 | 0.515 | 0.307 | 0.382 | 0.446 | 0.469 | 0.537 | 0.430 | 0.128 |
| 24 | 0.096 | 0.005 | 0.062 | 0.043 | 0.076 | 0.026 | 0.029 | 0.054 | 0.089 | 0.053 | 0.030 |

TABLE 5

| | |
|---|---|
| Control | Lonicera japonica |

| Parameters | Average | S.D | Average | S.D |
|---|---|---|---|---|
| Tₘₐₓ (hr) | 3.550 | 2.885 | 2.889 | 3.090 |
| Cₘₐₓ (μg/mL) | 0.921 | 0.238 | 0.736 | 0.184 |
| AUCₗₐₛₜ | 10.77 | 3.191 | 8.737 | 2.670 |
| (μg · hr/mL) | | | | |
| T_{1/2} (h) | 5.053 | 0.951 | 4.838 | 1.524 |

### Example 2

### Development of Complex Preparation of Therapeutic Agent for Lung Cancer Gefitinib (Iressa™) and Hot-Water Extract Lyophilisate of Lonicera japonica (LF): Effects of LF Complex Composition on Anticancer Effects of Gefitinib in NCI-H520 Non-Small Cell Lung Cancer Cell Xenograft Nude Mice

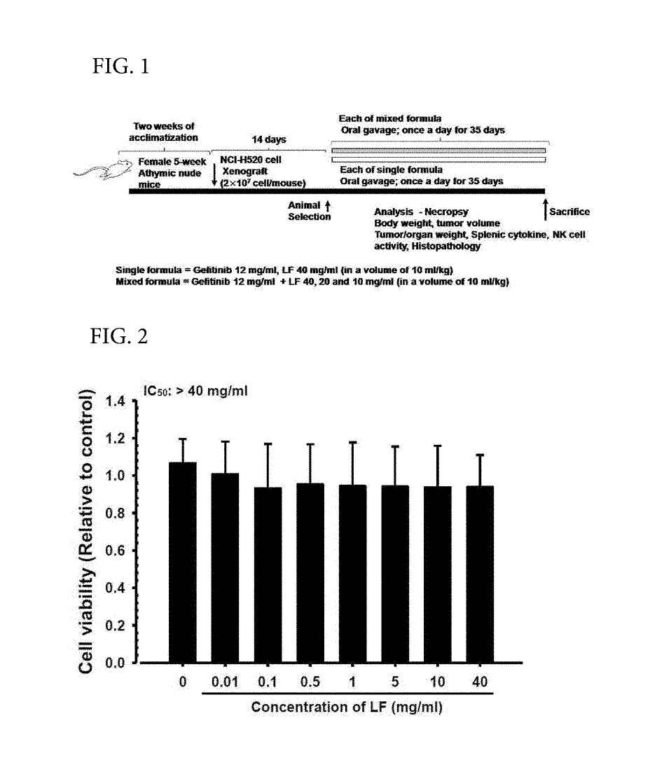

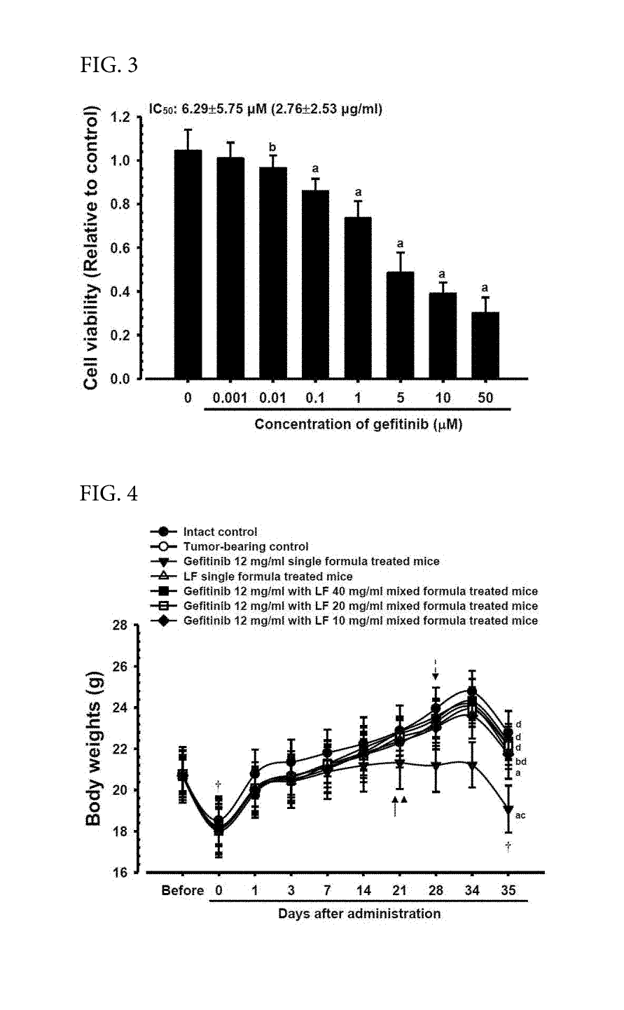

In the present Example 2, as an effort of Oriental and Western integrated medical studies of gefitinib and LF, effects of the LF complex composition on the anticancer effects of gefitinib were intended to be evaluated by using NCI-H520 cells of a representative human non-small cell lung squamous cell carcinoma (NSCLC) cell line. In the present Example 2, the cytotoxicity of LF and gefitinib for the NCI-H520 cell line was evaluated by a general MTT method, single compositions of 12 mg/ml of gefitinib and 400 mg/ml of LF and complex compositions of 12 mg/ml of gefitinib and 40, 20, or 10 mg/ml of LF were administered orally to athymic nude mice for 35 days from day 15 after the NCI-H520 lung cancer cell line was xenografted, and then all the experimental animals were sacrificed, and the anticancer and immune activity effects were each observed by observing the changes in body weight, tumor volume, tumor weight, weights of the immune organs (thymus and submandibular lymph node), serum interferon (IFN)-γ content, natural killer (NK) cell activity, and intrasplenic contents of tumor necrosis factor (TNF)-α, interleukin (IL)-1β, and IL-10 along with the histopathological changes of a tumor and lymph organs. Further, in order to observe the effects on tumor-related cachexia, a change in periovarian fat pad weight and a change in serum IL-6 content were observed, the changes in thickness of periovarian fat pad and diameter of adipocytes were each histopathologically observed, and in a tumor mass formed, the immunoreactivities of cleaved caspase-3 and poly(ADPribose) polymerase (PARP) as apoptotic markers, nitrotyrosine (NT) as an inducible nitric oxide synthases (iNOS)-related oxidative stress marker, 4-hydroxynonenal (4-HNE) as a lipid peroxidation marker, CD-31 and Ki-67 as angiogenic and cell proliferation markers, cyclooxygenase-2 (COX-2) as an inflammatory and angiogenic factor, and iNOS and TNF-α as cytokines related to immune activity were each observed by avidin-biotin-peroxidase (ABC)-based immunohistochemical methods. In the present Example 2, the results for the complex composition were compared with those of the group to which a single composition of 12 mg/ml of gefitinib was administered, and all the experimental materials were dissolved in sterile distilled water and orally administered at a dose of 10 ml/kg, which is a general oral administration dose, once a day for 35 days (Table 6, FIG. 1).

TABLE 6

| | | | |
|---|---|---|---|
| Group | Xenograft | Dose (concentrations) | Animal No. |

CIMI-16-02-01 G + LF PD: Effects on NCI-H520 cell xenograft nude mice

| | | | |
|---|---|---|---|
| Control | Saline | Vehicle 10 ml/kg | M01-M08 |
| Control | NCI-H520 cells | Vehicle 10 ml/kg | M09-M16 |
| Reference | NCI-H520 cells | Gefitinib single formula (12 mg/ml) | M17-M24 |
| Reference | NCI-H520 cells | LF single formula (40 mg/ml) | M25-M32 |
| Active | NCI-H520 cells | Gefitinib and LF (12 with 40 mg/ml) mixed | M33-M40 |
| | | formula | |
| Active | NCI-H520 cells | Gefitinib and LF (12 with 20 mg/ml) mixed | M41-M48 |
| | | formula | |
| Active | NCI-H520 cells | Gefitinib and LF (12 with 10 mg/ml) mixed | M49-M56 |
| | | formula | |

2.1. Experimental Method

Evaluation of Cytotoxicity

IC50, which is a concentration at which LF (0, 0.01, 0.1, 0.5, 1, 5, 10, and 40 mg/ml) and gefitinib (0, 0.001, 0.01, 0.1, 1, 5, 10, and 50 μM) suppressed the viability of NCI-H520 cells (1×10⁴ cells) to 50%, was evaluated by using a general MTT method.

Experimental Animals

A total of 110 five-week old female SPF/VAF CAnN.Cg-Foxn1 nu/CrljOri mice (OrientBio, Seongnam, Korea) were purchased, the animals with a constant weight were selected after 14 day acclimatization, HNCI-H520 cells were xenografted into the right hip subcutaneous site of each mouse, and then 14 days after tumor cells were xenografted, 8 nude mice per group were used in the present experiment based on tumor volume (504.02±120.50 mm³, ranging from 255.54 to 768.58 mm³) and body weight (tumor-xenografted group −20.73±0.97 g, 19.10 to 23.20 g), and a separate intact vehicle control consisting of 8 nude mice was also prepared based on body weight (20.73±1.21 g, 19.40 to 22.70 g).

Separation of Groups (Total 7 Groups; 8 Mice per Group)

(1) Intact control: Intact vehicle control

(2) TB control: Group to which sterile distilled water was administered after HCI-H520 tumor cells were xenografted

(3) G120: Group to which a single composition of 12 mg/ml of gefitinib was administered after tumor cells were xenografted

(4) LF400: Group to which a single composition of 40 mg/ml of LF was administered after tumor cells were xenografted

(5) G+LF400: Group to which a complex composition of 12 mg/ml of gefitinib and 40 mg/ml of LF was administered after tumor cells were xenografted

(6) G+LF200: Group to which a complex composition of 12 mg/ml of gefitinib and 20 mg/ml of LF was administered after tumor cells were xenografted

(7) G+LF100: Group to which a complex composition of 12 mg/ml of gefitinib and 10 mg/ml of LF was administered after tumor cells were xenografted

Tumor Cell Xenograft

NCI-H520 (American Type Culture Collection Center, Manassas, Va., USA) cells were subcultured and maintained in a 5% CO₂ incubator at 37° C. by using a RPMI 1640 (Gibco, Grand Island, N.Y., USA) medium supplemented with 10% fetal bovine serum (FBS), a tumor cell suspension was prepared so as to obtain a concentration of 1.0×10⁸ cell/ml, and a solid tumor mass was formed by xenografting 0.2 mL (2×10⁷ cell/mouse) of the NCI-H520 tumor cell suspension into the right dorsal hip dermis of each mouse. In the present Example 2, 14 days after the NCI-H520 lung cancer cell line was xenografted, experimental animals consisting of 8 mice per group were selected based on tumor volume (504.02±120.50 mm³, ranging from 255.54 to 68.58 mm³).

Drug Administration

In the group to which a single composition of gefitinib was administered, 12 mg of gefitinib (Suzhou Huihe Pharm Co., Ltd., Suzhou, China) was dissolved per ml of sterile distilled water, and in the LF single composition, 40 mg of LF was also dissolved per ml of sterile distilled water. Further, in the groups to which the complex compositions of gefitinib and LF were administered, LF at a concentration of each of 40, 20, and 10 mg/ml was additionally dissolved in the gefitinib composition at a concentration of 12 mg/ml. From day 15 after NCI-H520 lung cancer cells were xenografted, single compositions of 12 mg/ml of gefitinib and 40 mg/ml of LF and complex compositions of 12 mg/ml of gefitinib and 40, 20, or 10 mg/ml of LF were forcibly orally administered once daily for 35 days by using a 1-ml syringe to which a metal zonde was attached, and in the intact and tumor xenografted vehicle controls, only sterile distilled water, which is a medium, was orally administered by the same method for 35 days from day 15 after a tumor was xenografted. The dose of gefitinib was selected as 120 mg/kg, and a concentration of 40, 20, or 10 mg/ml of the complex composition of LF was set based on the results in Example 1.

Observation Items

IC50 (cytotoxicity), which is a concentration at which LF and gefitinib suppressed the viability of NCI-H520 cells to 50%, was evaluated by a general MTT method, and in the mice into which the NCI-H520 lung cancer cell line was xenografted, changes in effects of the LF complex composition on the anticancer and immune activities, and tumor-related cachexia were each evaluated (Tables 6 and 7, and FIG. 1).

(1) Anticancer effects: Changes in tumor volume, tumor weight, tumor cell volume in a formed mass, and apoptotic cell percentages, and changes in immunoreactivities of caspase-3, PARP, NT, 4-HNE, CD31, Ki-67, COX-2, iNOS, and TNF-α in the formed mass

(2) Immune activity effects: changes in weights of the immune organs (thymus and submandibular lymph node), serum interferon (IFN)-γ content, NK cell activity, and intrasplenic contents of TNF-α, interleukin (IL)-1β, and IL-10, histological changes of the immune organs, and change in immunoreactivity of TNF-α in a tumor mass and submandibular lymph node,

(3) Effects of suppressing tumor-related cachexia: Changes in body weight, periovarian fat pad weight, and serum IL-6 content and histological change in a periovarian fat pad

TABLE 7

| Antisera or detection kits | Code | Source | Dilution |
|---|---|---|---|
| Primary antisera* | | | |
| Anti-cleaved caspase-3 (Asp175) | 9661 | Cell Signaling Technology Inc. | 1:400 |
| polyclonal antibody | | Beverly, MA, USA | |
| Anti-cleaved PARP (h215) antibody | sc-23461 | Santa Cruz Biotechnology Inc. | 1:100 |
| | | Santa Cruz, CA, USA | |
| Anti-Nitrotyrosine antibody | 06-284 | Millipore, Temecula, CA, USA | 1:200 |
| Anti-4-Hydroxynonenal (4-HNE) | ab46545 | Abcam, Cambridge, UK | 1:100 |
| antibody | | | |
| Anti-CD31 (PECAM-1) antibody | 550274 | BD Bioscience, San Jose, CA, USA | 1:100 |
| Anti-Ki-67 (Sp6) antibody | ab16667 | Abcam, Cambridge, UK | 1:100 |
| Anti-cyclooxygenase (murine) | 160126 | Cayman Chemical., Ann Arbor, MI, | 1:200 |
| polyclonal antibody | | USA | |
| Anti-nitric oxide synthase2 (N- | sc-651 | Santa Cruz Biotechnology, Santa | 1:100 |
| 20) polyclonal antibody | | Cruz, CA, USA | |
| Anti-tumor necrosis factor-α | sc-52746 | Santa Cruz Biotechnology, Santa | 1:200 |
| antibody | | Cruz, CA, USA | |
| Detection kits | | | |
| Vectastain Elite ABC Kit | PK-6200 | Vector Lab. Inc., Burlingame, CA, | 1:50 |
| | | USA | |
| Peroxidse substrate kit | SK-4100 | Vector Lab. Inc., Burlingame, CA, | 1:50 |
| | | USA | |

2.2. Result Confirmation

2.2.1. Cytotoxicity

(1) Effects of LF on the Viability of NCI-H520 Cells

A significant change in the viability of NCI-H520 cells was not acknowledged in the groups treated with LF at all the seven concentrations (0.01, 0.1, 0.5, 1, 5, 10, and 40 mg/ml) as compared to the vehicle control (a treatment group at 0 mg/ml), so that IC50 was calculated as >40 mg/ml (FIG. 2).

The groups treated with LF at a concentration of 0.01, 0.1, 0.5, 1, 5, 10, and 40 mg/ml exhibited changes in the viability of NCI-H520 cells of −5.45, −12.44, −10.53, −11.39, −11.65, −12.00, and 11.82%, respectively, as compared to the vehicle control (a treatment group at 0 mg/ml).

(2) Effects of Gefitinib on the Viability of NCI-H520 Cells

A significant (p<0.01 or p<0.05) decrease in the viability of NCI-H520 cells began to be acknowledged from a group treated with gefitinib at 0.01 μM as compared to the vehicle control (a treatment group at 0 μM), so that IC50 was calculated as 6.29±5.75 μM (2.76±2.53 μg/ml).

The groups treated with gefitinib at a concentration of 0.001, 0.01, 0.1, 1, 5, 10, and 50 μM exhibited changes in the viability of NCI-H520 cells of −3.29, −7.47, −17.73, −29.55, −53.53, −62.71, and −71.11%, respectively, as compared to the vehicle control (a treatment group at 0 μM).

2.2.2. Changes in Body Weight and Body Weight Gain

A significant change in body weight in a tumor xenograft control was not acknowledged throughout the entire experimental period as compared to the intact vehicle control, but as compared to the intact vehicle control, each of a significant (p<0.01) decrease in actual body weight excluding the tumor weight and a significant (p<0.01) decrease in body weight gain for an administration period based on the actual body weight were acknowledged. In the groups to which a single composition of 12 mg/ml of gefitinib was administered, a significant (p<0.01) decrease in body weight was acknowledged from day 28 after administration, and significant (p<0.01) decreases in body weight and body weight gain were also acknowledged. Meanwhile, in the group to which the single composition of 40 mg/ml of LF was administered and the groups to which complex compositions of 12 mg/ml of gefitinib and 40, 20, and 10 mg/ml of LF were administered, significant (p<0.01 or p<0.05) increases in actual body weight and body weight gain were each acknowledged as compared to the tumor xenograft group, and in particular, in the groups to which complex compositions of 12 mg/ml of gefitinib and 40, 20, and 10 mg/ml of LF were administered, a significant (p<0.01 or p<0.05) increase in body weight began to be acknowledged from day 21 after administration as compared to the group to which the single composition of 12 mg/ml of gefitinib was administered, so that significant (p<0.01) increases in actual body weight and body weight gain were also each acknowledged (Table 8, FIG. 4).

On the final sacrifice date, for the actual body weight excluding the tumor weight, the tumor xenograft control exhibited a change of −15.10% as compared to the intact vehicle group, and the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −8.18, 6.37, 12.99, 10.16, and 7.28%, respectively, as compared to the tumor xenograft control.

For the body weight gain (35 days; the actual body weight excluding the tumor weight on the final sacrifice date—the body weight on the date when administration began) for the administration period based on the actual body weight, the tumor xenograft control exhibited a change of −71.82% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −133.07, 105.99, 220.07, 156.72, and 110.21%, respectively, as compared to the tumor xenograft control.

TABLE 8

| | Body weights (g) | | | |
|---|---|---|---|---|
| | At first | | Actual body | Body weight gains |
| Groups | administration | At sacrifice | weights* | (g) † |

Controls

| | | | | |
|---|---|---|---|---|
| Intact | 18.53 ± 1.15 | 22.78 ± 1.06 | 22.78 ± 1.06 | 4.25 ± 0.75 |
| TB | 18.14 ± 1.19 | 21.86 ± 0.84 | 19.34 ± 1.11a | 1.20 ± 0.63a |

Single formula treated

| | | | | |
|---|---|---|---|---|
| Gefitinib | 18.15 ± 1.42 | 19.08 ± 1.14ac | 17.75 ± 1.11ac | −0.40 ± 1.01ac |
| LF | 18.10 ± 1.23 | 22.19 ± 0.74e | 20.67 ± 0.93ade | 2.47 ± 0.93ade |

Gefitinib 12 mg/ml and LF mixed formula

| | | | | |
|---|---|---|---|---|
| 40 mg/ml | 18.01 ± 1.07 | 21.86 ± 0.93e | 21.86 ± 0.93ce | 3.83 ± 0.98ce |
| 20 mg/ml | 18.23 ± 0.94 | 22.15 ± 0.93e | 21.30 ± 0.96ace | 3.07 ± 1.12bce |
| 10 mg/ml | 16.23 ± 1.27 | 21.74 ± 1.20be | 20.74 ± 1.26ade | 2.52 ± 1.24ace |

2.2.3. Change in Tumor Volume

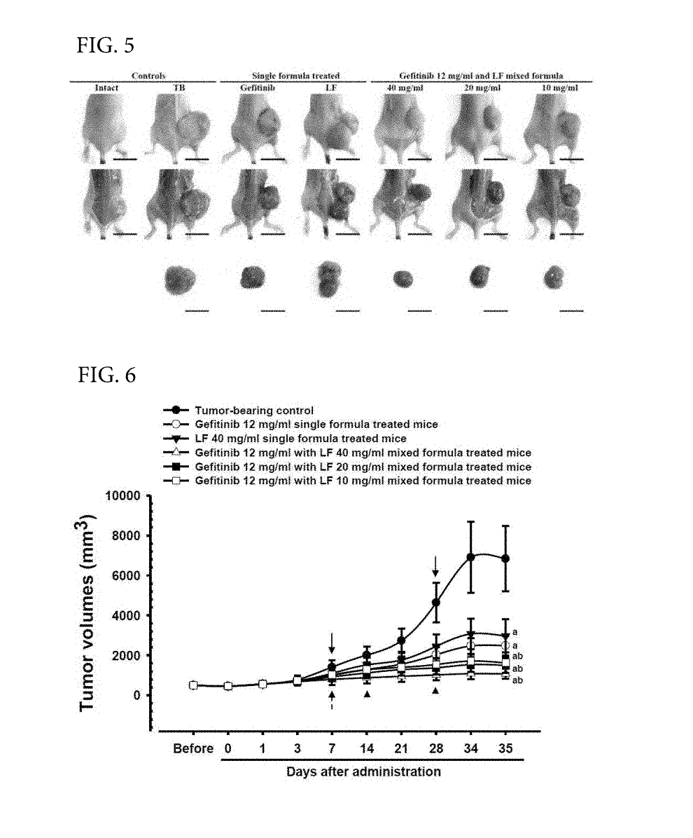

The groups to which the single compositions of 12 mg/ml of gefitinib and 40 mg/ml of LF were administered exhibited a significant (p<0.01 or p<0.05) decrease in tumor volume from days 7 and 28 after the beginning of administration, respectively, as compared to the tumor xenograft control, so that the groups also exhibited a significant (p<0.01) decrease in the amount of change in tumor volume, respectively for the administration period as compared to the tumor xenograft control. The groups to which the complex compositions of 12 mg/ml of gefitinib and 40, 20, or 10 mg/ml of LF were administered also exhibited a significant (p<0.01 or p<0.05) decrease in tumor volume from day 7 after the beginning of administration, respectively, as compared to the tumor xenograft control, so that the groups also exhibited a significant (p<0.01) decrease in the amount of change in tumor volume, respectively for the administration period as compared to the tumor xenograft control. In particular, in the groups to which complex compositions of 12 mg/ml of gefitinib and 40, 20, or 10 mg/ml of LF were administered, a significant (p<0.01 or p<0.05) decrease in tumor volume was acknowledged from day 14, 28, or 28 after the beginning of administration, respectively, as compared to the group to which the single composition of 12 mg/ml of gefitinib was administered, and the groups also exhibited a significant (p<0.01) decrease in the amount of change in tumor volume for the administration period, respectively as compared to the group to which the single composition of 12 mg/ml of gefitinib was administered (Table 9, and FIGS. 5 and 6).

For the amount of change in tumor volume (5 weeks; tumor volume on the final sacrifice date—tumor volume on the date when administration began) for the drug administration period, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −68.18, −60.57, −90.34, −83.66, and −81.70% as compared to the tumor xenograft group.

TABLE 9

| | Tumor volume (mm³) | | |
|---|---|---|---|
| | First | | |
| | administration | | Changes (mm³) |
| Groups | [A] | Sacrifice [B] | [B − A] |

Control

| | | | |
|---|---|---|---|
| TB | 463.07 ± 141.04 | 6831.41 ± 1631.47 | 6368.35 ± 1518.76 |

Single formula treated

| | | | |
|---|---|---|---|
| Gefitinib | 459.88 ± 129.50 | 2486.04 ± 480.24a | 2026.16 ± 394046a |
| LF | 464.67 ± 126.37 | 2975.46 ± 828.31a | 2510.79 ± 811.60a |

Gefitinib 12 mg/ml and LF mixed formula

| | | | |
|---|---|---|---|
| 40 mg/ml | 458.02 ± 138.14 | 1073.19 ± 248.57ab | 615.17 ± 217.09ab |
| 20 mg/ml | 458.76 ± 94.50 | 1499.41 ± 381.63ab | 1040.65 ± 298.05ab |
| 10 mg/ml | 458.86 ± 87.47 | 1624.03 ± 274.27ab | 1165.17 ± 233.36ab |

2.2.4. Change in Tumor Weight

In all the single or combination administration groups of gefitinib and LF, significant (p<0.01) decreases in tumor relative and absolute weights were acknowledged as compared to the tumor xenograft group, and in particular, in the groups to which the complex compositions of 12 mg/ml of gefitinib and 40, 20, or 10 mg/ml of LF were administered, significant (p<0.01) decreases in tumor relative and absolute weights were acknowledged, respectively, as compared to the group to which the single composition of 12 mg/ml of gefitinib was administered (Tables 10 and 11, and FIG. 5).

The groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change in tumor absolute weight of −47.72, −35.89, −80.06, −66.36, and −60.64%, respectively, and a change in tumor relative weight by −40.22, −36.79, −80.46, −66.85, and −60.33%, respectively, as compared to the tumor xenograft group.

2.2.5. Change in Splenic Weight

In the tumor xenograft control, significant (p<0.01) decreases in splenic absolute and relative weights were each acknowledged, but in the group to which the single composition of 40 mg/ml of LF was administered and the groups to which the complex compositions of 12 mg/ml of gefitinib and 40, 20, or 10 mg/ml of LF were administered, a significant (p<0.01) increase in splenic weight was acknowledged, respectively, as compared to the tumor xenograft group, and in particular, in the groups to which the complex compositions of 12 mg/ml of gefitinib and 40, 20, or 10 mg/ml of LF were administered, significant (p<0.01 or p<0.05) increases in splenic absolute and relative weights were acknowledged, respectively, as compared to the group to which the single composition of 12 mg/ml of gefitinib was administered. Meanwhile, in the group to which the single composition of 12 mg/ml of gefitinib was administered, significant changes in splenic absolute and relative weights were not acknowledged as compared to the tumor xenograft group (Tables 10 and 11).

For the splenic absolute weight, the tumor xenograft control exhibited a change of −41.96% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −1.46, 31.04, 56.25, 47.71, and 31.67%, respectively, as compared to the tumor xenograft control.

For the splenic relative weight, the tumor xenograft control exhibited a change of −39.59% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 12.85, 29.33, 52.88, 45.68, and 32.48%, respectively, as compared to the tumor xenograft control.

TABLE 10

| | | | | |
|---|---|---|---|---|
| | | | Submandibular | Periovarian fat |
| Groups | Tumor mass | Spleen | lymph node | pad |

Controls

| | | | | |
|---|---|---|---|---|
| Intact | | 0.103 ± 0.010 | 0.006 ± 0.002 | 0.038 ± 0.010 |
| TB | 2.527 ± 0.485 | 0.060 ± 0.010a | 0.003 ± 0.001a | 0.008 ± 0.002e |

Single formula treated

| | | | | |
|---|---|---|---|---|
| Gefitinib | 1.321 ± 0.191g | 0.059 ± 0.010a | 0.003 ± 0.001a | 0.008 ± 0.002e |
| LF | 1.620 ± 0.266g | 0.079 ± 0.013acd | 0.006 ± 0.002acd | 0.021 ± 0.003egh |

Gefitinib 12 mg/ml and LF mixed formula

| | | | | |
|---|---|---|---|---|
| 40 mg/ml | 0.504 ± 0.120gh | 0.094 ± 0.005bcd | 0.007 ± 0.001cd | 0.027 ± 0.004fgh |
| 20 mg/ml | 0.850 ± 0.142gh | 0.089 ± 0.006acd | 0.006 ± 0.001acd | 0.023 ± 0.005egh |
| 10 mg/ml | 0.995 ± 0.097gh | 0.079 ± 0.007acd | 0.006 ± 0.001acd | 0.021 ± 0.003egh |

TABLE 11

| | | | | |
|---|---|---|---|---|
| | | | Submandibular | Periovarian fat |
| Groups | Tumor mass | Spleen | lymph node | pad |

Controls

| | | | | |
|---|---|---|---|---|
| Intact | | 0.455 ± 0.052 | 0.036 ± 0.009 | 0.165 ± 0.046 |
| TB | 11.602 ± 2.419 | 0.275 ± 0.045a | 0.014 ± 0.005a | 0.038 ± 0.008f |

Single formula treated

| | | | | |
|---|---|---|---|---|
| Gefitinib | 6.935 ± 0.963h | 0.310 ± 0.050a | 0.017 ± 0.006a | 0.042 ± 0.011f |
| LF | 7.333 ± 1.391h | 0.355 ± 0.064ac | 0.026 ± 0.007ace | 0.094 ± 0.015fhi |

Gefitinib 12 mg/ml and LF mixed formula

| | | | | |
|---|---|---|---|---|
| 40 mg/ml | 2.268 ± 0.588hi | 0.420 ± 0.031cd | 0.031 ± 0.006cd | 0.119 ± 0.020ghi |
| 20 mg/ml | 3.846 ± 0.677hi | 0.400 ± 0.0267bcd | 0.028 ± 0.005bcd | 0.105 ± 0.023fhi |
| 10 mg/ml | 4.603 ± 0.659hi | 0.364 ± 0.035ace | 0.026 ± 0.006acd | 0.097 ± 0.016fhi |

2.2.6. Change in Submandibular Lymph Node Weight

In the tumor xenograft control, significant (p<0.01) decreases in submandibular lymph node absolute and relative weights were each acknowledged, but in the group to which the single composition of 40 mg/ml of LF was administered and the groups to which the complex compositions of 12 mg/ml of gefitinib and 40, 20, and 10 mg/ml of LF were administered, a significant (p<0.01) increase in splenic weight was acknowledged, respectively, as compared to the tumor xenograft group, and in particular, in the groups to which the complex compositions of 12 mg/ml of gefitinib and 40, 20, or 10 mg/ml of LF were administered, significant (p<0.01 or p<0.05) increases in submandibular lymph node absolute and relative weights were acknowledged, respectively, as compared to the group to which the single composition of 12 mg/ml of gefitinib was administered. Meanwhile, in the group to which the single composition of 12 mg/ml of gefitinib was administered, a significant change in submandibular lymph node was not acknowledged as compared to the tumor xenograft group (Tables 10 and 11).

For the submandibular lymph node absolute weight, the tumor xenograft control exhibited a change of −62.12% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 4.00, 76.00, 124.00, 100.00, and 84.00%, respectively, as compared to the tumor xenograft control.

For the submandibular lymph node relative weight, the tumor xenograft control exhibited a change of −60.41% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 19.67, 72.81, 119.12, 97.55, and 84.81%, respectively, as compared to the tumor xenograft control.

2.2.7. Change in Periovarian Fat Pad Weight

In the tumor xenograft control, significant (p<0.01) decreases in periovarian fat pad absolute and relative weights were each acknowledged, but in the group to which the single composition of 40 mg/ml of LF was administered and the groups to which the complex compositions of 12 mg/ml of gefitinib and 40, 20, or 10 mg/ml of LF were administered, a significant (p<0.01) increase in periovarian fat pad weight was acknowledged, respectively, as compared to the tumor xenograft group, and in particular, in the groups to which the complex compositions of 12 mg/ml of gefitinib and 40, 20, or 10 mg/ml of LF were administered, a significant (p<0.01) increase in periovarian fat pad weight was acknowledged as compared to the group to which the single composition of 12 mg/ml of gefitinib was administered. Meanwhile, in the group to which the single composition of 12 mg/ml of gefitinib was administered, significant changes in accumulated periovarian fat pad absolute and relative weights were not acknowledged as compared to the tumor xenograft group (Tables 10 and 11).

For the periovarian fat pad absolute weight, the tumor xenograft control exhibited a change of −78.07% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −1.52, 151.52, 222.73, 180.30, and 154.55%, respectively, as compared to the tumor xenograft control.

For the periovarian fat pad relative weight, the tumor xenograft control exhibited a change of −77.12% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 12.22, 148.01, 215.54, 176.45, and 156.64%, respectively, as compared to the tumor xenograft control.

2.2.8. Changes in Serum IL-6 and IFN-γ Contents

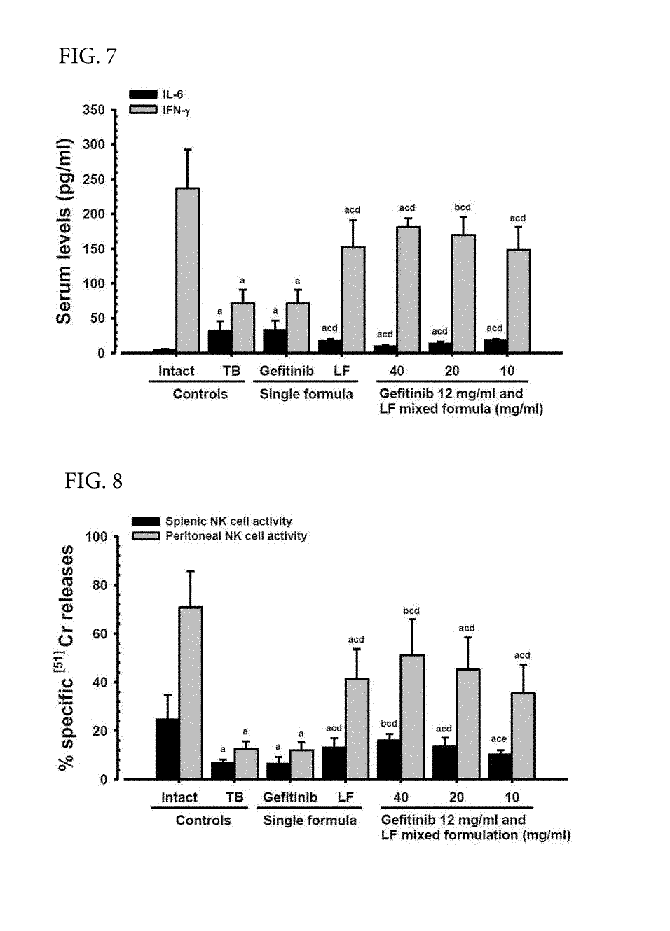

In the tumor xenograft control, a significant (p<0.01) increase in serum IL-6 content and a significant (p<0.001) decrease in serum IFN-γ content were each acknowledged as compared to the intact vehicle control, but in the group to which the single composition of 40 mg/ml of LF was administered and the groups to which the complex compositions of 12 mg/ml of gefitinib and 40, 20, or 10 mg/ml of LF were administered, a significant (p<0.01) decrease in serum IL-6 content and a significant (p<0.01) increase in serum IFN-γ content were each acknowledged, respectively as compared to the tumor xenograft control, and in particular, in the groups to which 12 mg/ml of gefitinib and 40, 20, and 10 mg/ml of LF were administered, a significant (p<0.01) decrease in serum IL-6 content and a significant (p<0.01) increase in serum IFN-γ content were each acknowledged, respectively, as compared to the group to which the single composition of 12 mg/ml of gefitinib was administered. Meanwhile, in the group to which the single composition of 12 mg/ml of gefitinib was administered, changes in serum IL-6 and IFN-γ contents were not acknowledged as compared to the tumor xenograft control (FIG. 7).

For the serum IL-6 content, the tumor xenograft control exhibited a change of 588.02% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 2.09, −47.12, −69.58, −58.77, and −44.98%, respectively, as compared to the tumor xenograft control.

For the serum IFN-γ content, the tumor xenograft control exhibited a change of −64.42% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −0.62, 112.66, 152.89, 137.80, and 107.50%, respectively, as compared to the tumor xenograft control.

2.2.9. Change in NK Cell Activity

In the tumor xenograft control, significant (p<0.01) decreases in splenic and peritoneal NK cell activities were each acknowledged, but in the group to which the single composition of 40 mg/ml of LF was administered and the groups to which the complex compositions of 12 mg/ml of gefitinib and 40, 20, or 10 mg/ml of LF were administered, significant (p<0.01) increases in splenic and peritoneal NK cell activities were acknowledged, respectively, as compared to the tumor xenograft group, and in particular, in the groups to which the complex compositions of 12 mg/ml of gefitinib and 40, 20, or 10 mg/ml of LF were administered, significant (p<0.01 or p<0.05) increases in splenic and peritoneal NK cell activities were acknowledged, respectively, as compared to the group to which the single composition of 12 mg/ml of gefitinib was administered. Meanwhile, in the group to which the single composition of 12 mg/ml of gefitinib was administered, significant changes in splenic and peritoneal NK cell activities were not acknowledged as compared to the tumor xenograft control (FIG. 8).

For the splenic NK cell activity, the tumor xenograft control exhibited a change of −72.20% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −6.16, 89.45, 133.99, 96.42, and 48.83%, respectively, as compared to the tumor xenograft control.

For the peritoneal NK cell activity, the tumor xenograft control exhibited a change of −82.16% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −4.12, 227.87, 305.00, 257.98, and 181.66%, respectively, as compared to the tumor xenograft control.

Change in Contents of Splenic Cytokines

In the tumor xenograft control, significant (p<0.01) decreases in content of splenic TNF-α, IL-1β, and IL-10 were each acknowledged, but in the group to which the single composition of 40 mg/mg of LF was administered and the groups to which the complex compositions of 12 mg/ml of gefitinib and 40, 20, or 10 mg/ml of LF were administered, a significant (p<0.01) increase in contents of splenic cytokines was acknowledged, respectively, as compared to the tumor xenograft group, and in particular, in the groups to which the complex compositions of 12 mg/ml of gefitinib and 40, 20, or 10 mg/ml of LF were administered, significant (p<0.01) increases in contents of splenic TNF-α, IL-1β, and IL-10 were acknowledged, respectively, as compared to the group to which the single composition of 12 mg/ml of gefitinib was administered. Meanwhile, in the group to which the single composition of 12 mg/ml of gefitinib was administered, a significant change in contents of splenic cytokines was not acknowledged as compared to the tumor xenograft group (Table 12).

For the content of splenic TNF-α, the tumor xenograft control exhibited a change of −78.05% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −4.79, 138.33, 162.03, 132.13, and 103.41%, respectively, as compared to the tumor xenograft control.

For the content of splenic IL-1β, the tumor xenograft control exhibited a change of −84.71% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −3.08, 337.99, 437.14, 393.42, and 250.94%, respectively, as compared to the tumor xenograft control.

For the content of splenic IL-10, the tumor xenograft control exhibited a change of −80.16% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −7.27, 162.11, 210.10, 158.68, and 105.43%, respectively, as compared to the tumor xenograft control.

TABLE 12

| | | | |
|---|---|---|---|
| | Tumor | | |
| Groups | necrosis factor-α | Interleukin-1β | Interleukin-10 |

Controls

| | | | |
|---|---|---|---|
| Intact | 125.01 ± 35.29 | 52.79 ± 12.36 | 127.15 ± 25.83 |
| TB | 27.43 ± 13.54a | 8.07 ± 1.76d | 25.22 ± 11.52a |

Single formula treated

| | | | |
|---|---|---|---|
| Gefitinib | 26.12 ± 11.32a | 7.82 ± 2.32d | 23.39 ± 12.60a |
| LF | 65.39 ± 11.38abc | 35.35 ± 10.63efg | 66.12 ± 17.06abc |

Gefitinib 12 mg/ml and LF mixed formula

| | | | |
|---|---|---|---|
| 40 mg/ml | 71.89 ± 15.88abc | 43.35 ± 10.37fg | 78.22 ± 24.00abc |
| 20 mg/ml | 63.68 ± 13.57abc | 39.83 ± 11.22fg | 65.25 ± 15.22abc |
| 10 mg/ml | 55.81 ± 12.61abc | 28.33 ± 10.32dfg | 51.82 ± 11.75abc |

2.2.11. Histological Change

(1) Histopathological Change of Tumor Mass

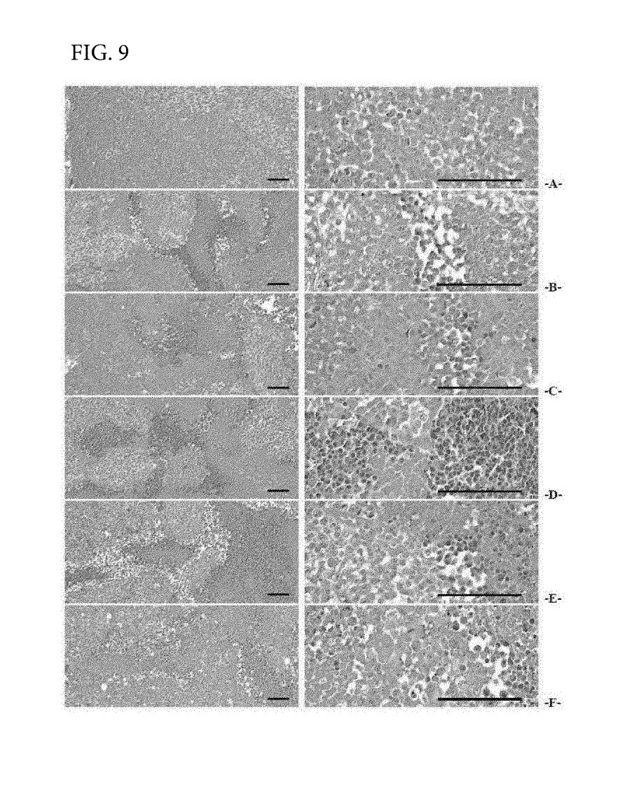

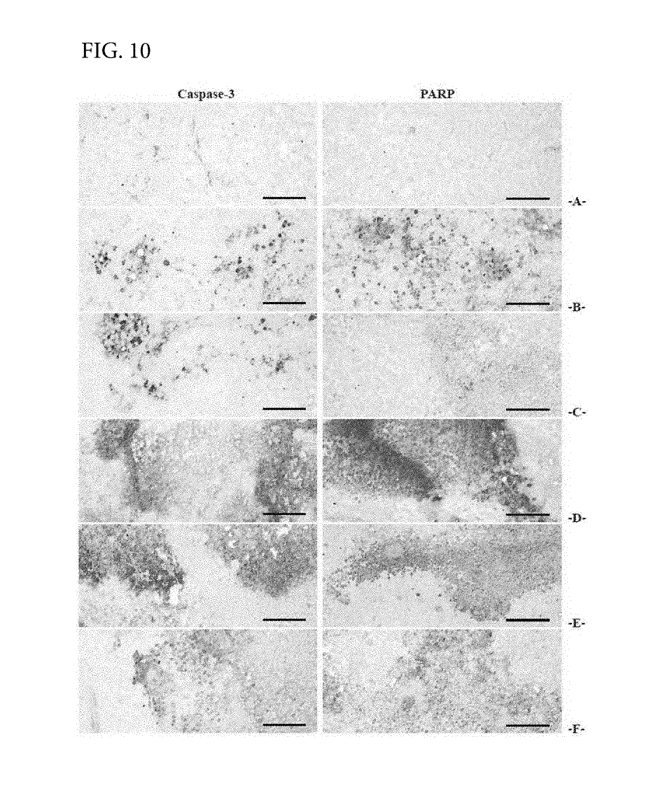

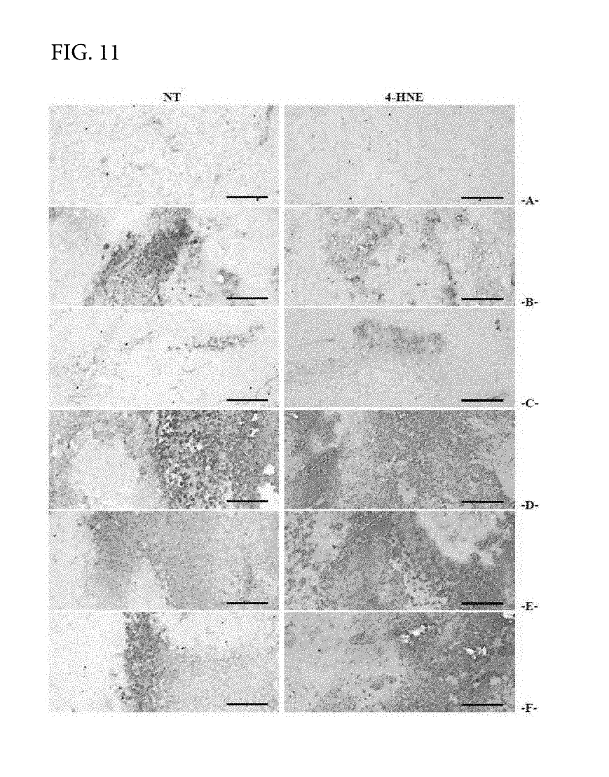

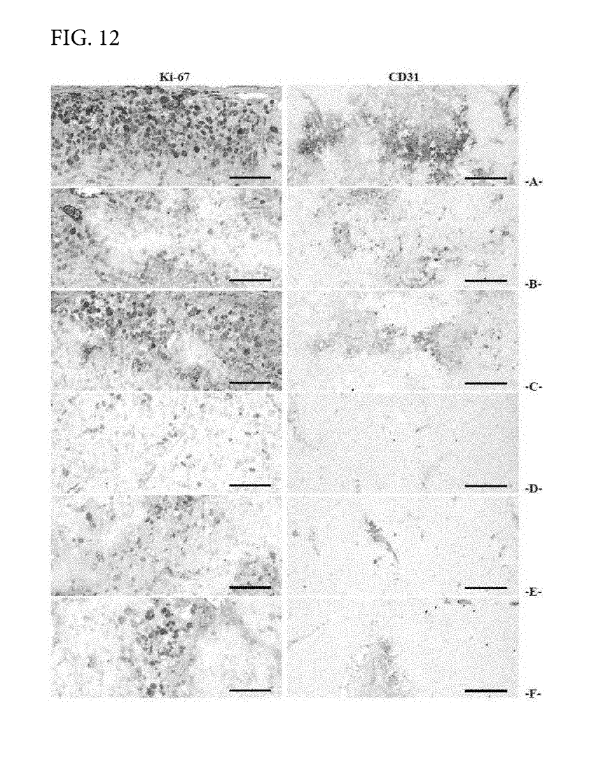

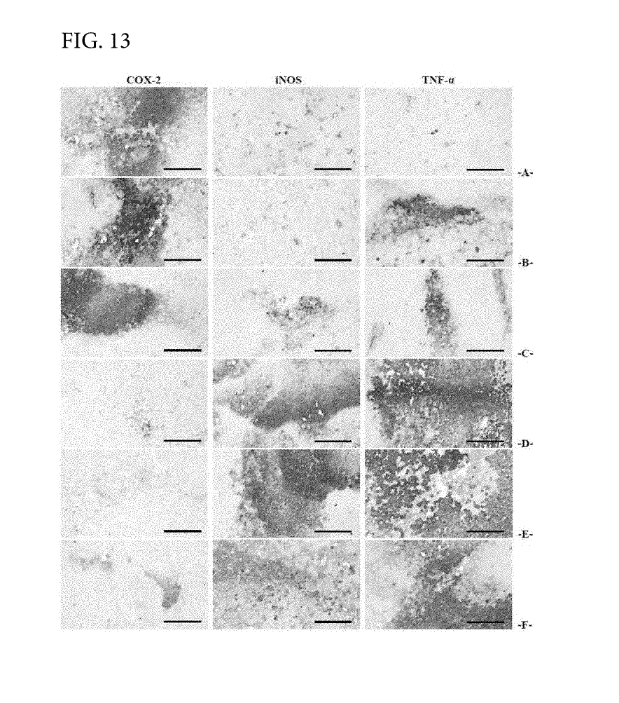

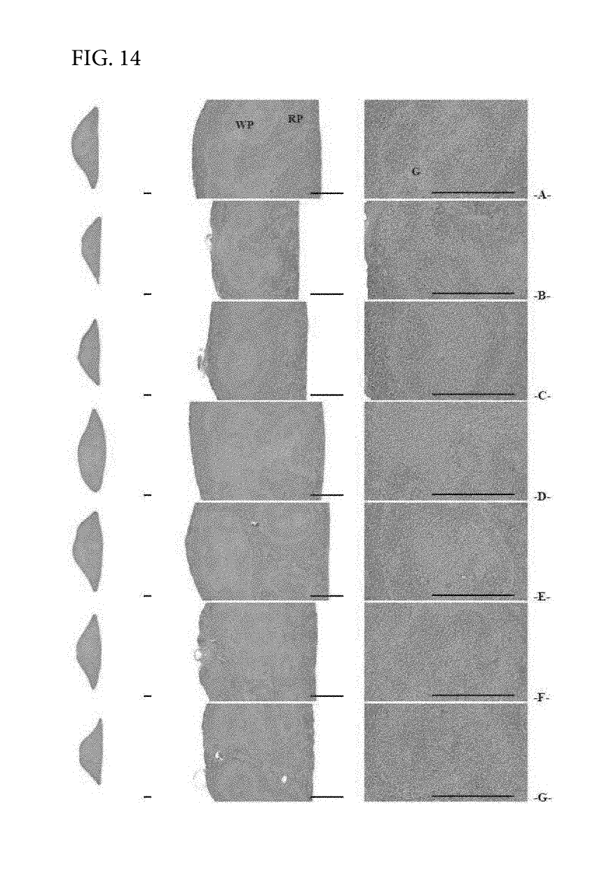

The tumor xenograft control compactly consists of NCI-H520 lung cancer cells which were relatively well differentiated, cytoplasmic eosinophilia and pyknosis due to apoptosis were acknowledged in extremely few cells, and mitosis was also frequently observed. Meanwhile, in the groups to which the single compositions of 12 mg/ml of gefitinib and 40 mg/ml of LF were administered and the groups to which the complex compositions of 40, 20, and 10 mg/ml of LF and 12 mg/ml of gefitinib were administered, a significant (p<0.01) decrease in the proportion of occupying NCI-H520 cells was acknowledged, respectively as compared to the tumor xenograft control, and in particular, in the groups to which the complex compositions of 12 mg/ml of gefitinib and 40, 20, or 10 mg/ml of LF were administered, a significant (p<0.01) decrease in the proportion of occupying tumor cells was also acknowledged, respectively as compared to the group to which the single composition of 12 mg/ml of gefitinib was administered (Table 13, FIG. 9). Further, in the group to which the single composition of 40 mg/ml of LF was administered and all the groups to which the complex compositions of 12 mg/ml of gefitinib and 40, 20, and 10 mg/ml of LF were administered, significant (p<0.01) increases in the numbers of caspase-3, PARP, NT, 4-HNE, iNOS, and TNF-α immunoreactive cells in a tumor mass and significant (p<0.01) decreases in the numbers of Ki-67, CD31, and COX-2 immunoreactive cells were acknowledged, as compared to the tumor xenograft control, and in particular, in the groups to which the complex compositions of 12 mg/ml of gefitinib and 40, 20, or 10 mg/ml of LF were administered, significant (p<0.01) decreases in the numbers of caspase-3, PARP, NT, 4-HNE, iNOS, and TNF-α immunoreactive cells and significant (p<0.01) increases in the numbers of Ki-67, CD31, and COX-2 immunoreactive cells were also acknowledged, respectively as compared to the group to which the single composition of 12 mg/ml of gefitinib was administered. Meanwhile, as compared to the tumor xenograft control, significant changes in the numbers of iNOS and COX-2 immunoreactive cells in a tumor mass were not acknowledged in the group to which 120 mg/kg of gefitinib was administered (Table 13, FIGS. 10 to 13).

TABLE 13

| | | Single formula | | Gefitinib 12 mg/ml and LF | | |
|---|---|---|---|---|---|---|
| Groups | TE | treated | | mixed formula | | |
| Items | control | Gefitinib | LF | 40 mg/ml | 20 mg/ml | 10 mg/ml |
| Tumor cell | 76.56 ± 10.04 | 54.48 ± 12.96a | 63.79 ± 11.82a | 23.63 ± 6.31ab | 32.23 ± 10.00ab | 35.77 ± 8.65ab |
| volume | | | | | | |
| (%/mm2) | | | | | | |

Immunoreactive cell percentages (%/tumor cells)

| | | | | | | |
|---|---|---|---|---|---|---|
| Cleaved | 5.55 ± 2.55 | 25.77 ± 5.73d | 21.30 ± 3.62df | 53.69 ± 15.42de | 46.25 ± 13.77de | 40.80 ± 10.61de |
| caspase-3 | | | | | | |
| PARP | 2.88 ± 1.08 | 22.27 ± 10.20d | 18.00 ± 10.02d | 63.06 ± 16.18de | 52.25 ± 13.92de | 47.88 ± 12.12de |
| Nitrotyrosine | 7.02 ± 2.21 | 32.93 ± 10.64d | 17.32 ± 3.16de | 66.73 ± 16.04de | 58.97 ± 12.52de | 48.82 ± 11.15df |
| 4- | 2.83 ± 1.44 | 29.12 ± 8.55a | 25.08 ± 12.65a | 63.76 ± 13.43ab | 52.27 ± 10.64ab | 45.43 ± 10.87ab |
| hydroxynonenal | | | | | | |
| Ki-67 | 77.59 ± 12.69 | 45.34 ± 7.63a | 55.16 ± 9.40a | 22.01 ± 10.59ab | 26.21 ± 11.37ab | 30.00 ± 11.02ab |
| CD31 | 50.94 ± 11.25 | 29.90 ± 5.37a | 36.97 ± 7.75ac | 12.07 ± 4.94ab | 16.03 ± 3.75ab | 22.61 ± 2.83ac |
| Cyclooxygenase- | 53.10 ± 10.69 | 56.48 ± 12.30 | 34.94 ± 12.82ab | 12.46 ± 6.33ab | 16.45 ± 7.72ab | 21.01 ± 7.26ac |
| 2 | | | | | | |
| INOS | 6.05 ± 1.92 | 5.34 ± 1.44 | 31.97 ± 10.87de | 48.99 ± 12.01de | 40.93 ± 14.79de | 27.79 ± 10.90de |
| Tumor necrosis | 5.42 ± 2.46 | 26.06 ± 7.96d | 223.31 ± 4.77d | 67.73 ± 13.90de | 59.16 ± 17.46de | 49.19 ± 13.32de |
| factor-a | | | | | | |

For the proportion of the tumor tissue occupied by tumor cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −30.68, −18.81, −69.67, −58.97, and −54.46%, respectively, as compared to the tumor xenograft control.

For the proportion of the tumor tissue occupied by caspase-3 immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 364.72, 284.15, 868.17, 734.02, and 635.80%, respectively, as compared to the tumor xenograft control.

For the proportion of the tumor tissue occupied by PARP immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 673.85, 525.41, 2,091.31, 1,715.90, and 1,564.07%, respectively, as compared to the tumor xenograft control.

For the proportion of the tumor tissue occupied by NT immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 368.85, 146.65, 850.21, 739.69, and 595.21%, respectively, as compared to the tumor xenograft control.

For the proportion of the tumor tissue occupied by 4-HNE immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 928.20, 785.39, 2,150.84, 1,745.23, and 1,503.80%, respectively, as compared to the tumor xenograft control.

For the proportion of the tumor tissue occupied by Ki-67 immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −41.57, −28.92, −71.63, −66.22, and −61.34%, respectively, as compared to the tumor xenograft control.

For the proportion of the tumor tissue occupied by CD31 immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −41.30, −27.41, −76.31, −64.60, and −55.61%, respectively, as compared to the tumor xenograft control.

For the proportion of the tumor tissue occupied by COX-2 immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 6.37, −34.20, −76.53, −69.02, and −60.42%, respectively, as compared to the tumor xenograft control.

For the proportion of the tumor tissue occupied by iNOS immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −11.83, 428.04, 709.32, 576.07, and 359.10%, respectively, as compared to the tumor xenograft control.

For the proportion of the tumor tissue occupied by TNF-α immunoreactive cells, the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 417.98, 311.95, 1,150.50, 992.34, and 808.24%, respectively, as compared to the tumor xenograft control.

(2) Histopathological Change of Spleen

In the tumor xenograft control, atrophy characterized by a remarkable decrease in lymphocytes of the splenic white pulp portion was acknowledged as compared to the intact vehicle control, so that significant (p<0.01) decreases in splenic thickness, white pulp diameter, and white pulp number were each acknowledged. Meanwhile, in the group to which the single composition of 40 mg/ml of LF was administered and the groups to which the complex compositions of 12 mg/ml of gefitinib and 40, 20, or 10 mg/ml of LF were administered, significant (p<0.01) and remarkable increases in spleen thickness, white pulp diameter, and white pulp number were histopathologically acknowledged, respectively as compared to the tumor xenograft control, and in particular, in the groups to which the complex compositions of 12 mg/ml of gefitinib and 40, 20, or 10 mg/ml of LF were administered, significant (p<0.01) increases in spleen thickness, white pulp diameter, and white pulp number were also acknowledged, respectively as compared to the group to which the single composition of 12 mg/ml of gefitinib was administered. Meanwhile, in the group to which the single composition of 12 mg/ml of gefitinib was administered, significant changes in spleen thickness, white pulp diameter, and white pulp number were acknowledged, respectively as compared to the tumor xenograft control (Table 14, FIG. 14).

TABLE 14

| | | | |
|---|---|---|---|
| | Total thickness | White pulp | White pulp diameters |
| Groups | (μm/central regions) | numbers (/mm2) | (μm/white pulp) |

Controls

| | | | |
|---|---|---|---|
| Intact | 1700.51 ± 228.44 | 15.63 ± 3.42 | 714.48 ± 111.59 |
| TB | 1109.96 ± 220.01a | 5.88 ± 1.46e | 316.52 ± 54.24e |

Single formula treated

| | | | |
|---|---|---|---|
| Gefitinib | 1097.00 ± 237.39a | 6.13 ± 1.81e | 334.72 ± 47.47e |
| LF | 1509.78 ± 207.77cd | 12.63 ± 2.33fgh | 505.28 ± 105.71egh |

Gefitinib 12 mg/ml and LF mixed formula

| | | | |
|---|---|---|---|
| 40 mg/ml | 1686.45 ± 218.14cd | 14.63 ± 2.56gh | 630.69 ± 116.00gh |
| 20 mg/ml | 1505.39 ± 125.94cd | 13.63 ± 1.60gh | 516.19 ± 100.87egh |
| 10 mg/ml | 1436.06 ± 134.69bcd | 11.63 ± 1.06egh | 458.93 ± 41.06egh |

For the total splenic thickness, the tumor xenograft control exhibited a change of −34.73% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −1.17, 36.02, 51.94, 35.63, and 29.38%, respectively, as compared to the tumor xenograft control.

For the splenic white pulp number, the tumor xenograft control exhibited a change of −62.40% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 4.26, 114.89, 148.94, 131.91, and 97.87%, respectively, as compared to the tumor xenograft control.

For the splenic white pulp diameter, the tumor xenograft control exhibited a change of −55.70% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 5.74, 59.64, 99.26, 63.08, and 44.99%, respectively, as compared to the tumor xenograft control.

(3) Histopathological Change of Submandibular Lymph Node

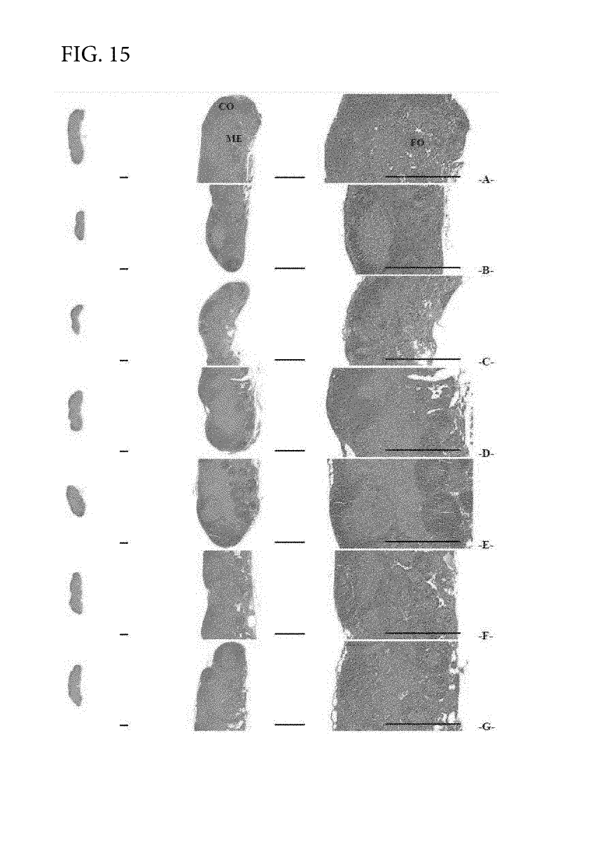

In the tumor xenograft control, a finding on atrophy caused by a remarkable decrease in submandibular lymph node cortex was acknowledged, so that significant (p<0.01) decreases in submandibular lymph node total and cortex thicknesses and cortex lymphoid cell follicle numbers are acknowledged, respectively. Meanwhile, in the group to which the single composition of 40 mg/ml of LF was administered and the groups to which the complex compositions of 12 mg/ml of gefitinib and 40, 20, or 10 mg/ml of LF were administered, significant (p<0.01) increases in submandibular lymph node total and cortex thicknesses and cortex lymphoid cell follicle numbers were histopathologically acknowledged, respectively, as compared to the tumor xenograft control, and in particular, in the groups to which the complex compositions of 12 mg/ml of gefitinib and 40, 20, or 10 mg/ml of LF were administered, significant (p<0.01) increases in submandibular lymph node total and cortex thicknesses and cortex lymphoid cell follicle numbers were also acknowledged, respectively, as compared to the group to which the single composition of 12 mg/ml of gefitinib was administered. In the group to which the single composition of 12 mg/ml of gefitinib was administered, significant changes in submandibular lymph node total and cortex thicknesses and cortex lymphoid cell follicle numbers were not acknowledged as compared to the tumor xenograft control (Table 15, FIG. 15).

TABLE 15

| | | | |
|---|---|---|---|
| | | Cortex | |
| | | lymphoid cell | |
| | Total thickness | follicle numbers | Cortex thickness |
| Groups | (μm/central regions) | (/mm2) | (μm/lymph node) |

Controls

| | | | |
|---|---|---|---|
| Intact | 920.08 ± 104.09 | 22.88 ± 4.52 | 503.48 ± 144.02 |
| TB | 484.73 ± 116.37a | 6.50 ± 2.07a | 167.58 ± 35.84a |

Single formula treated

| | | | |
|---|---|---|---|
| Gefitinib | 441.48 ± 114.06a | 7.50 ± 2.39a | 155.92 ± 34.47a |
| LF | 715.88 ± 109.43abc | 15.75 ± 2.43abc | 335.57 ± 53.58abc |

Gefitinib 12 mg/ml and LF mixed formula

| | | | |
|---|---|---|---|
| 40 mg/ml | 826.54 ± 96.64abc | 17.50 ± 1.77abc | 380.42 ± 75.75abc |
| 20 mg/ml | 751.07 ± 101.85abc | 15.38 ± 2.45abc | 351.34 ± 67.18abc |
| 10 mg/ml | 705.43 ± 122.62abc | 13.63 ± 2.26abc | 271.70 ± 56.51abc |

For the submandibular lymph node total thickness, the tumor xenograft control exhibited a change of −47.32% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −8.92, 47.69, 70.51, 54.94, and 45.53%, respectively, as compared to the tumor xenograft control.

For the cortex lymphoid cell follicle numbers, the tumor xenograft control exhibited a change of −71.58% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 15.38, 142.31, 169.23, 136.54, and 109.62%, respectively, as compared to the tumor xenograft control.

For the submandibular lymph node cortex thickness, the tumor xenograft control exhibited a change of −66.72% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −6.96, 100.24, 127.00, 109.65, and 62.13%, respectively, as compared to the tumor xenograft control.

(4) Histopathological Change of Periovarian Fat Pad

In the tumor xenograft control, atrophy characterized by a remarkable decrease in size of white adipocytes was acknowledged as compared to the intact vehicle control, so that significant (p<0.01) decreases in accumulated periovarian fat pad thickness and average white adipocyte diameter are acknowledged, respectively.

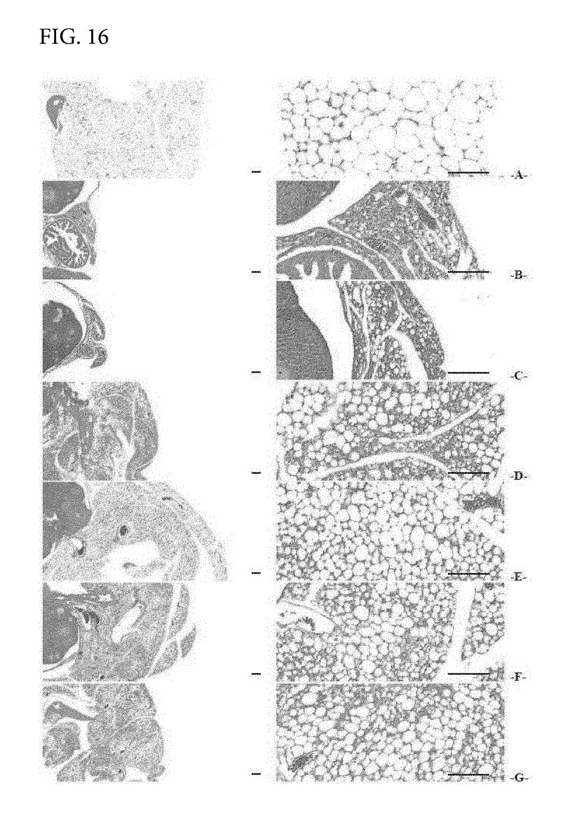

Meanwhile, in the group to which the single composition of 40 mg/ml of LF was administered and the groups to which the complex compositions of 12 mg/ml of gefitinib and 40, 20, or 10 mg/ml of LF were administered, significant (p<0.01) increases in accumulated fat thickness and average white adipocyte diameter were histopathologically acknowledged, respectively, as compared to the tumor xenograft control, and in particular, in the groups to which the complex compositions of 12 mg/ml of gefitinib and 40, 20, or 10 mg/ml of LF were administered, significant (p<0.01) increases in accumulated fat thickness and average white adipocyte diameter were also acknowledged, respectively, as compared to the group to which the single composition of 12 mg/ml of gefitinib was administered. Meanwhile, in the group to which the single composition of 12 mg/ml of gefitinib was administered, significant changes in accumulated periovarian fat pad tissue thickness and average white adipocyte diameter were not acknowledged (Table 16, FIG. 16).

TABLE 16

| | | |
|---|---|---|
| | Total thickness (μm/central | |
| Groups | regions) | White adipocyte diameters (μm) |

Controls

| | | |
|---|---|---|
| Intact | 2042.50 ± 396.92 | 63.38 ± 11.52 |
| TB | 294.21 ± 100.89 | 12.95 ± 2.51 |

Single formula treated

| | | |
|---|---|---|
| Gefitinib | 261.47 ± 125.47 | 13.52 ± 3.46 |
| LF | 838.19 ± 139.15 | 27.00 ± 2.65 |

Gefitinib 12 mg/ml and LF mixed formula

| | | |
|---|---|---|
| 40 mg/ml | 1229.53 ± 257.23 | 35.77 ± 5.15 |
| 20 mg/ml | 904.87 ± 207.24 | 27.91 ± 7.23 |
| 10 mg/ml | 838.55 ± 138.51 | 24.76 ± 6.04 |

For the accumulated periovarian fat pad thickness, the tumor xenograft control exhibited a change of −85.60% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of −11.13, 184.89, 317.91, 207.56, and 185.02%, respectively, as compared to the tumor xenograft control.

For the average periovarian white fat pad cell diameter, the tumor xenograft control exhibited a change of −79.57% as compared to the intact vehicle group, but the groups to which the single compositions of 12 mg/ml of gefitinib and 40 mg/ml of LF were administered and the groups to which the complex compositions of 12 mg/ml of gefitinib and 40, 20, or 10 mg/ml of LF were administered exhibited a change of 4.41, 108.54, 176.21, 115.52, and 91.19%, respectively, as compared to the tumor xenograft control.

As can be confirmed from the results in the present Example 2, for LF and gefitinib, the IC₅₀ for NCI-H520 cells was calculated as >40 mg/ml and 6.29±5.75 μM (2.76±2.53 μg/ml), respectively, significant decreases in splenic and submandibular lymph node weights, serum IFN-γ content, intrasplenic contents of TNF-α, IL-1β, and IL-10, and spleen cell and peritoneal macrophage activities were acknowledged along with a finding on histopathological atrophy caused by decreases in splenic and submandibular lymph node lymphocytes, decreases in actual body weight excluding the tumor weight and body weight gain based on the actual body weight were also acknowledged, and an increase in serum IL-6 content, a decrease in periovarian fat pad weight, and atrophy of accumulated periovarian fat pad tissue were histopathologically acknowledged. Accordingly, it is determined that after the tumor xenograft, typical tumor-related immune suppression and cachexia phenomena are caused. Meanwhile, decreases in tumor mass volume and weight by administering the single composition of 12 mg/ml of gefitinib were acknowledged along with a decrease in the proportion of tumor cells in a tumor mass in a histopathological examination, increases in the numbers of caspase-3, PARP, NT, 4-HNE, and TNF-α immunoreactive cells in a tumor mass and decreases in the numbers of Ki-67 and CD31 immunoreactive cells were acknowledged, but it was observed that COX-2 and iNOS immunoreactivities in tumor tissue, tumor-related cachexia (changes in body weight, periovarian fat pad, and serum IL-6 content), and immune suppression (changes in spleen and submandibular lymph node weights, serum IFN-γ content, NK cell activity, and intrasplenic contents of TNF-α, IL-1β, and IL-10, and histological changes of the immune organs) were not particularly affected. Meanwhile, remarkable decreases in immune activity and tumor-related cachexia were acknowledged as compared to the tumor xenograft control, but it was observed that the anticancer effect against the tumor mass itself was relatively lower than those of the group to which gefitinib was administered. In addition, in the groups to which the complex compositions of 12 mg/ml of gefitinib and 40, 20, or 10 mg/ml of LF were administered, a finding on significant and remarkable suppression of anticancer, immune activity, and tumor-related cachexia was acknowledged as compared to the tumor xenograft control, and in particular, a significant increase in anticancer effects, which is dependent on the concentration of LF constituting the complex, was acknowledged, and significant and remarkable increases in effects of decreasing the immune activity and cachexia were also exhibited in a manner which is concentration-dependent on LF constituting the complex as compared to the group to which the single composition of 12 mg/ml of gefitinib was administered.

Accordingly, it was determined that the LF complex composition remarkably enhanced anticancer effects of gefitinib through already well-known immune regulation effects without affecting the bioavailability of gefitinib, and effectively controlled tumor-related cachexia through an increase in antioxidant activity, so that it is expected that the complex composition of gefitinib and LF can provide a therapeutic method which is very useful for cooperative diagnosis by Oriental and Western medicine and treatment of patients with lung cancer. Further, even in the groups to which the complex composition of 10 mg/ml of LF and gefitinib were administered, increases in tumor-related cachexia suppression effects and anticancer activity caused by significant and remarkable immune activity and activity of the antioxidant defense system were also acknowledged as compared to the group to which gefitinib was administered alone, so that when a complex composition of LF at a concentration of 10 mg/ml or more is prepared, the immune activity can clearly increase the anticancer effects of gefitinib and control the tumor-related cachexia.

### Example 3

### Development of Complex Preparation of Therapeutic Agent for Lung Cancer Gefitinib (Iressa™) and Hot-Water Extract Lyophilisate of Lonicera japonica (LF): Effects of Reducing Toxicity of Gefitinib According to LF Complex Composition

3.1. Separation of Experimental Animals and Groups

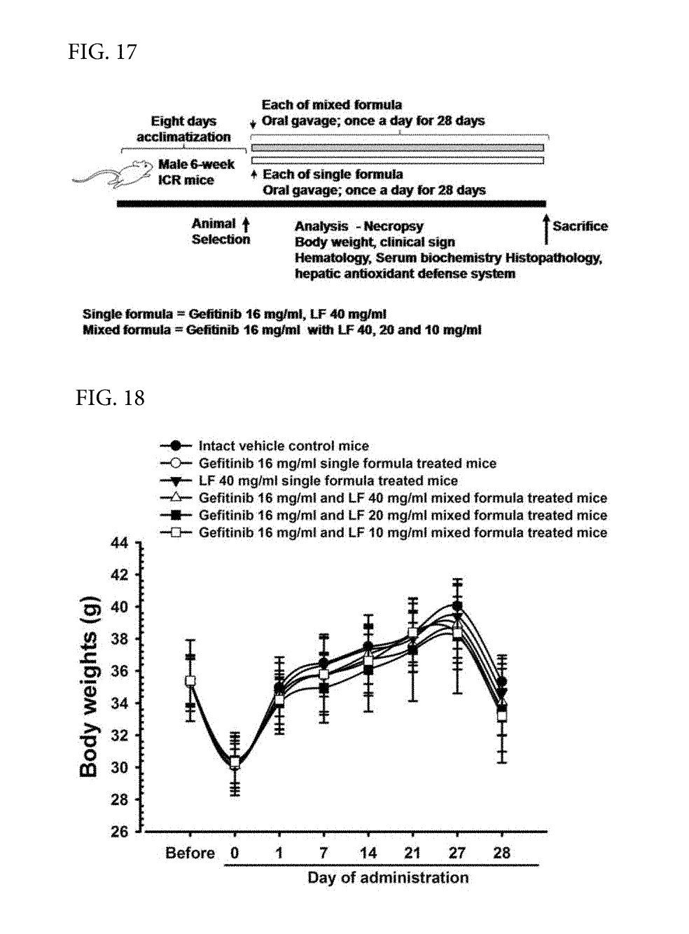

After SPF/VAF Outbred CrljOri:CD1[ICR] male mice (OrientBio, Seongnam, Korea) as male ICR mice were acclimatized for 8 days, 7 mice per group were selected based on body weight (average: 35.35±1.64 g, 31.60 to 38.40 g), and the mice were separated into 6 groups and used (Table 17, FIG. 17).

Total 6 Groups (Including Vehicle Control); 7 Mice Per Group (Used Total: 42 Mice)

(GOM) vehicle control, (G1M) group to which a single composition of 16 mg/ml of gefitinib is administered, (G2M) group to which a single composition of 40 mg/ml of LF is administered, (G3M) group to which a complex composition of 16 mg/ml of gefitinib and 40 mg/ml of LF is administered, (G4M) group to which a complex composition of 16 mg/ml of gefitinib and 20 mg/ml of LF is administered, (G5M) group to which a complex composition of 16 mg/ml of gefitinib and 10 mg/ml of LF is administered

TABLE 17

| | | | |
|---|---|---|---|
| | | | Animal |
| Groups | Sex | Dose (mg/kg) | No. |

CIMI-16-02-01 G + LF TX: Mouse repeated oral dose toxicity test

| | | | |
|---|---|---|---|
| Control | Male | Distilled water 10 ml/kg | M01~M07 |
| Reference | Male | Gefitinib 16 mg/ml single formula | M08~M14 |
| | | (160 mg/kg) | |
| Reference | Male | LF 40 mg/ml single formula (400 mg/ml) | M15~M21 |
| Active | Male | Gefitinib and LF (160 and 400 mg/kg) | M22~M28 |
| | | as mixed formula | |
| Active | Male | Gefitinib and LF (160 and 200 mg/kg) | M29~M35 |
| | | as mixed formula | |
| Active | Male | Gefitinib and LF (160 and 100 mg/kg) | M36~M42 |
| | | as mixed formula | |

LF = Lonicerae Flos (dried flower parts of Lonicera japonica Thunb., Caprifoliaceae) aqueous extracted lyophilized powders, test material

3.2. Experimental Purpose, Administration Method, and Dose

Single compositions of 16 mg/ml of gefitinib and 40 mg/ml of LF and complex compositions containing 16 mg/ml of gefitinib and 40, 20, or 10 mg/ml of LF for oral administration (oral gavage; orally administered at a dose of 10 ml/kg, which is a general oral dose for a rodent, once daily for 28 days by using sterile distilled water as a solvent) were each orally administered once daily for 28 days by using a zonde attached to a 1-ml syringe, and only sterile distilled water, which is a medium, was administered orally to the vehicle control at the same dose once daily for 28 days in order to apply the same stress to the vehicle control according to the oral administration and the correction.

In the group to which a single composition of gefitinib was administered, 16 mg of gefitinib (Suzhou Huihe Pharm Co., Ltd., Suzhou, China) was dissolved per ml of sterile distilled water, and in the LF single composition, 40 mg of LF was dissolved per ml of sterile distilled water. Further, in the groups to which the complex compositions of gefitinib and LF were administered, LF at a concentration of each of 40, 20, and 10 mg/ml was additionally dissolved in the gefitinib composition at a concentration of 16 mg/ml (Table 17, FIG. 17).

In Example 3, the effects of reducing the toxicity of gefitinib according to the complex composition of LF were intended to be evaluated by using the male mice. The dose of gefitinib was set at 160 mg/kg, which is 4-times greater than 40 mg/kg that is a minimum dose known to exhibit cytotoxicity in rats during continuous administration for 28 days, and the concentration of the complex composition of LF was set at 40, 20, and 10 mg/ml (400, 200, or 100 mg/kg as the dose).

The observation items of 4-week mortality, clinical symptoms, change in body weight, and autopsy findings are as follows.

Day 28; mortality, clinical symptoms, change in body weight, autopsy findings, hematological (14 items; Table 18) and hematochemical (20 items; Table 19) changes, histopathological changes (23 organs: brain cerebrum, cerebellum and bulbar, heart, thymus, lung, testis, epididymis, kidney, adrenal gland, spleen, liver, pancreas, alimentary tract esophagus, fundic portion, pyloric portion, duodenum, jejunum, ileum, cecum, colon and rectum, submandibular lymph node), and changes in content of liver lipid peroxidation and antioxidant defense system glutathione (GSH), and catalase (CAT) and superoxide dismutase (SOD) activities.

TABLE 18

| Hematology Item | | | |
|---|---|---|---|
| Abbreviations | Full name | Units | Methods |
| 1. RBC | Red blood cell count | M/μL | Laser optical (Flow cytometry) |
| 2. HGB | Hemoglobin concentration | g/dl | Cyanmethemoglobin method |
| 3. HCT | Hematocrit | % | Calculated from Item 1 and 4 |
| 4. MCV | Mean corpuscular volume | fL | Laser optical (Flow cytometry) |
| 5. MCH | Mean corpuscular hemoglobin | pg | Calculated from Item 1 and 2 |
| 6. MCHC | Mean corpuscular hemoglobin | g/dL | Calculated from Item 2 and 3 |
| | concentration | | |
| 7. PLT | Platelet count | K/μL | Laser optical (Flow cytometry) |
| 8. RET | Reticulocyte count | ea/1000 | Laser optical with |
| | | | cytochemical reaction |
| 9. WEC | White blood cell count | K/μL | Laser optical with |
| | | | cytochemical reaction |
| | Differential counts of white blood | | |
| | cells | | |
| 10. NEU % | Percentages of neutrophils | % | Perox optical with chemical |
| | | | reaction |
| 11. LYM % | Percentages of lymphocytes | % | Perox optical with chemical |
| | | | reaction |
| 12. MON % | Percentages of monocytes | % | Perox optical with chemical |
| | | | reaction |
| 13. EOS % | Percentages of eosinophils | % | Perox optical with chemical |
| | | | reaction |
| 14. BAS % | Percentages of basophils | % | Perox optical with chemical |
| | | | reaction |

TABLE 19

| Hematology Items | | | |
|---|---|---|---|
| Abbreviations | Full name | Units | Methods |
| 1. AST | Aspartate aminotransferase | IU/L | UV-Rate method |
| 2. ALT | Alanine aminotransferase | IU/L | UV-Rate method |
| 3. ALP | Alkaline phosphatase | IU/L | P-NPP method |
| 4. BUN | Blood urea nitrogen | mg/dL | Urease-UV method |
| 5. CRE | Creatinine | mg/dL | Jaffe method |
| 6. GLU | Glucose | mg/dL | Enzyme method |
| 7. CHO | Total cholesterol | mg/dL | Enzyme method |
| 8. PRO | Total protein | g/dL | Biuret method |
| 9. CPK | Creatine phosphokinase | IU/L | UV-Rate method |
| 10. ALB | Albumin | g/dL | BCG method |
| 11. BIL | Total bilirubin | mg/dL | Jendrassick-cleghorn method |
| 12. Globulin | Globulin | g/dL | Calculated from Item 8 and 10 |
| 13. A/G | Albumin/globulin ratio | Ratio | Calculated from Item 10 and 12 |
| 14. IP | Inorganic phosphorus | mg/dL | UV method |
| 15. Ca | Calcium | mg/dL | OCPC method |
| 16. TG | Triglyceride | mg/dL | Enzyme method |
| 17. LDH | Lactate dehydrogenase | IU/L | UV-Rate method |
| 18. Na | Sodium | mmol/L | Electrode method |
| 19. K | Potassium | mmol/L | Electrode method |
| 20. Cl | Chloride | mmol/L | Electrode method |

3.3. Experimental Results

3.3.1. Mortality and Clinical Symptoms

As a result of the present Example 3, no cases of death related to the administration of the experimental material were acknowledged for an experimental period of 28 days, so that a final autopsy was carried out on all the experimental animals of all the experimental groups (7/7; 100%) (Table 20).

Furthermore, as a result of the present Example 3, no clinical symptoms related to the administration of the experimental material were observed for an experimental period of 28 days (Table 21).

TABLE 20

| | | | |
|---|---|---|---|
| | | At termination (at end | |
| Periods | Days of treatment | of 28 days of | |
| Groups | Periods (Day 0a~27) | administration) | Total* |

Vehicle control

| | | | |
|---|---|---|---|
| Distilled water | 0 | 0 | 0/7 (0%) |

Gefitinib single formula

| | | | |
|---|---|---|---|
| 16 mg/ml | 0 | 0 | 0/7 (0%) |

LF single formula

| | | | |
|---|---|---|---|
| 40 mg/ml | 0 | 0 | 0/7 (0%) |

Gefitinib 16 mg/ml and LF mixed formula

| | | | |
|---|---|---|---|
| 40 mg/ml | 0 | 0 | 0/7 (0%) |
| 20 mg/ml | 0 | 0 | 0/7 (0%) |
| 10 mg/ml | 0 | 0 | 0/7 (0%) |

TABLE 21

| | | |
|---|---|---|
| Signs | | Clinical signs |
| Groups | Normal appearance | Any abnormal signs |

Vehicle control

| | | |
|---|---|---|
| Distilled water | 7/7 (100%) | 0/7 (0%) |

Gefitinib single formula

| | | |
|---|---|---|
| 16 mg/ml | 7/7 (100%) | 0/7 (0%) |

LF single formula

| | | |
|---|---|---|
| 40 mg/ml | 7/7 (100%) | 0/7 (0%) |

Gefitinib 16 mg/ml and LF mixed formula

| | | |
|---|---|---|
| 40 mg/ml | 7/7 (100%) | 0/7 (0%) |
| 20 mg/ml | 7/7 (100%) | 0/7 (0%) |
| 10 mg/ml | 7/7 (100%) | 0/7 (0%) |

3.3.2. Changes in Body Weight and Body Weight Gain

A significant (p<0.01) decrease in body weight gain was acknowledged throughout the entire period of administration (Day 0 to 28) in the group to which the single composition of 16 mg/ml of gefitinib was administered as compared to the vehicle group, the group to which the complex composition of 16 mg/ml of gefitinib and 20 mg/ml of LF was administered exhibited a significant (p<0.01) decrease in body weight gain for Day 0 to Day 14 as compared to the vehicle control, the group to which the complex composition of 16 mg/ml of gefitinib and 10 mg/ml of LF was administered a significant (p<0.01 or p<0.05) decrease in body weight gain for each of Day 14 to Day 27 and Day 0 to Day 28, as compared to the vehicle control, but in all the groups to which the complex compositions were administered, significant increases in body weight and body weight gain were not acknowledged throughout the entire experimental period as compared to the group to which the single composition of 16 mg/ml of gefitinib was administered, and the groups to which the single composition of 40 mg/ml of LF was administered and the complex composition of 16 mg/ml of gefitinib and 40 mg/ml of LF was administered, significant changes in body weight and body weight gain were also not acknowledged through the entire experimental period as compared to the vehicle control (Table 22; FIG. 18).

TABLE 22

| Intervals | Intervals | | |
|---|---|---|---|
| Groups | Day 0*~Day 14 | Day 14~Day 27 | Day 0~Day 28** |

Vehicle control

| | | | |
|---|---|---|---|
| Distilled water | 7.44 ± 1.15 | 2.51 ± 0.56 | 5.26 ± 1.33 |

Gefitinib single formula

| | | | |
|---|---|---|---|
| 16 mg/ml | 6.53 ± 0.65 | 1.80 ± 0.62 | 3.26 ± 0.57b |

LF single formula

| | | | |
|---|---|---|---|
| 40 mg/ml | 7.26 ± 1.52 | 2.04 ± 1.60 | 4.54 ± 2.06 |

Gefitinib 16 mg/ml and LF mixed formula

| | | | |
|---|---|---|---|
| 40 mg/ml | 6.96 ± 1.16 | 1.86 ± 0.70 | 3.96 ± 1.18 |
| 20 mg/ml | 5.64 ± 1.49a | 2.06 ± 1.02 | 3.17 ± 2.29 |
| 10 mg/ml | 6.29 ± 0.79 | 1.73 ± 0.59c | 2.86 ± 0.93b |

3.3.3. Change in Organ Weight

In the group to which the single composition of 16 mg/ml of gefitinib was administered, significant (p<0.01) increases in spleen, liver, and submandibular lymph node absolute and relative weights were acknowledged, respectively, as compared to the vehicle control, but in all the groups to which the complex compositions of 10, 20, or 40 mg/ml of LF and 16 mg/ml of gefitinib were administered, significant (p<0.01) decreases in spleen, liver, and submandibular lymph node absolute and relative weights were acknowledged in a manner which is concentration-dependent on LF constituting the complex, respectively, as compared to the group to which the single composition of 16 mg/ml of gefitinib was administered. Meanwhile, in the group to which the single composition of 40 mg/ml of LF was administered, significant changes in relative and absolute organ weights were not acknowledged as compared to the vehicle control, and the group to which the single composition of 40 mg/ml of LF was administered exhibited significant (p<0.01) decreases in spleen, liver, and submandibular lymph node absolute and relative weights, respectively, as compared to the group to which the single composition of 16 mg/ml of gefitinib was administered. Meanwhile, even in all the groups to which the single composition of 16 mg/ml of gefitinib was administered and the complex compositions of the three types of gefitinib and LF were administered, no significant changes in lung, heart, thymus, kidney, adrenal gland, testis, pancreas, brain, and epididymis absolute and relative weights were not acknowledged as compared to the vehicle control (Tables 23 and 24).

TABLE 23

| | Principal organs | | | | | |
|---|---|---|---|---|---|---|
| Organs | | | | Kidney | Adrenal | |
| Groups | Lung | Heart | Thymus | L | GL | Spleen |
| Vehicle | 0.190 ± 0.015 | 0.167 ± 0.012 | 0.043 ± 0.009 | 0.295 ± 0.035 | 0.004 ± 0.001 | 0.085 ± 0.004 |
| control | | | | | | |

Gefitinib single formula

| | | | | | | |
|---|---|---|---|---|---|---|
| 16 | 0.181 ± 0.015 | 0.156 ± 0.013 | 0.048 ± 0.009 | 0.283 ± 0.030 | 0.005 ± 0.003 | 0.153 ± 0.014c |
| mg/ml | | | | | | |

LF single formula

| | | | | | | |
|---|---|---|---|---|---|---|
| 40 | 0.186 ± 0.015 | 0.159 ± 0.012 | 0.045 ± 0.013 | 0.299 ± 0.033 | 0.004 ± 0.002 | 0.097 ± 0.019d |
| mg/ml | | | | | | |

Gefitinib 16 mg/ml and LF mixed formula

| 40 | 0.192 ± 0.015 | 0.164 ± 0.015 | 0.042 ± 0.007 | 0.299 ± 0.032 | 0.004 ± 0.002 | 0.119 ± 0.013cd |
|---|---|---|---|---|---|---|
| mg/ml | | | | | | |
| 20 | 0.185 ± 0.010 | 0.162 ± 0.021 | 0.041 ± 0.013 | 0.291 ± 0.073 | 0.004 ± 0.003 | 0.125 ± 0.07cd |
| mg/ml | | | | | | |
| 10 | 0.190 ± 0.011 | 0.163 ± 0.010 | 0.042 ± 0.009 | 0.289 ± 0.032 | 0.004 ± 0.002 | 0.130 ± 0.007cd |
| mg/ml | | | | | | |
| Groups | | | Pancreas | | Epididymis | |
| Organs | Testis L | Liver | S | Brain | L | LN L |
| Vehicle | 0.121 ± 0.008 | 1.226 ± 0.0068 | 0.194 ± 0.026 | 0.483 ± 0.019 | 0.049 ± 0.006 | 0.003 ± 0.001 |
| control | | | | | | |

Gefitinib single formula

| | | | | | | |
|---|---|---|---|---|---|---|
| 16 | 0.117 ± 0.016 | 1.727 ± 0.065a | 0.203 ± 0.029 | 0.482 ± 0.017 | 0.046 ± 0.07 | 0.015 ± 0.003a |
| mg/ml | | | | | | |

LF single formula

| | | | | | | |
|---|---|---|---|---|---|---|
| 40 | 0.120 ± 0.004 | 1.206 ± 0.052b | 0.195 ± 0.032 | 0.476 ± 0.018 | 0.048 ± 0.004 | 0.003 ± 0.002b |
| mg/ml | | | | | | |

Gefitinib 16 mg/ml and LF mixed formula

| | | | | | | |
|---|---|---|---|---|---|---|
| 40 | 0.118 ± 0.012 | 1.373 ± 0.097ab | 0.204 ± 0.037 | 0.482 ± 0.021 | 0.044 ± 0.005 | 0.010 ± 0.002ab |
| mg/ml | | | | | | |
| 20 | 0.114 ± 0.019 | 1.448 ± 0.098ab | 0.204 ± 0.020 | 0.486 ± 0.015 | 0.048 ± 0.003 | 0.010 ± 0.002ab |
| mg/ml | | | | | | |
| 10 | 0.114 ± 0.015 | 1.480 ± 0.051ab | 0.208 ± 0.028 | 0.484 ± 0.015 | 0.046 ± 0.003 | 0.011 ± 0.001ab |
| mg/ml | | | | | | |

TABLE 24

| | Principal organs | | | | | |
|---|---|---|---|---|---|---|
| Organs | | | | Kidney | Adrenal | |
| Groups | Lung | Heart | Thymus | L | GL | Spleen |
| Vehicle | 0.574 ± 0.060 | 0.505 ± 0.056 | 0.130 ± 0.023 | 0.892 ± 0.132 | 0.012 ± 0.004 | 0.256 ± 0.018 |
| control | | | | | | |

Gefitinib single formula

| | | | | | | |
|---|---|---|---|---|---|---|
| 16 | 0.547 ± 0.039 | 0.472 ± 0.061 | 0.147 ± 0.034 | 0.857 ± 0.119 | 0.013 ± 0.010 | 0.461 ± 0.029a |
| mg/ml | | | | | | |

LF single formula

| | | | | | | |
|---|---|---|---|---|---|---|
| 40 | 0.562 ± 0.065 | 0.480 ± 0.049 | 0.136 ± 0.040 | 0.910 ± 0.166 | 0.013 ± 0.008 | 0.291 ± 0.055b |
| mg/ml | | | | | | |

Gefitinib 16 mg/ml and LF mixed formula

| 40 | 0.581 ± 0.055 | 0.497 ± 0.053 | 0.127 ± 0.024 | 0.875 ± 0.102 | 0.012 ± 0.005 | 0.360 ± 0.037ab |
|---|---|---|---|---|---|---|
| mg/ml | | | | | | |
| 20 | 0.559 ± 0.058 | 0.492 ± 0.023 | 0.124 ± 0.038 | 0.882 ± 0.235 | 0.010 ± 0.009 | 0.377 ± 0.036ab |
| mg/ml | | | | | | |
| 10 | 0.573 ± 0.023 | 0.492 ± 0.024 | 0.125 ± 0.0 | 0.873 ± 0.096 | 0.012 ± 0.006 | 0.391 ± 0.028ab |
| mg/ml | | | | | | |
| Groups | | | Pancreas | | Epididymis | |
| Organs | Testis L | Liver | S | Brain | L | LN L |
| Vehicle | 0.365 ± 0.034 | 3.712 ± 0.358 | 0.588 ± 0.110 | 1.463 ± 0.135 | 0.147 ± 0.020 | 0.009 ± 0.004 |
| control | | | | | | |

Gefitinib single formula

| | | | | | | |
|---|---|---|---|---|---|---|
| 16 | 0.355 ± 0.062 | 5.229 ± 0.510a | 0.616 ± 0.128 | 1.459 ± 0.110 | 0.139 ± 0.025 | 0.045 ± 0.008a |
| mg/ml | | | | | | |

LF single formula

| | | | | | | |
|---|---|---|---|---|---|---|
| 40 | 0.363 ± 0.029 | 3.646 ± 0.287b | 0.593 ± 0.117 | 1.440 ± 0.126 | 0.145 ± 0.012 | 0.009 ± 0.006b |
| mg/ml | | | | | | |

Gefitinib 16 mg/ml and LF mixed formula

| | | | | | | |
|---|---|---|---|---|---|---|
| 40 | 0.363 ± 0.029 | 4.160 ± 0.517ab | 0.617 ± 0.129 | 1.459 ± 0.159 | 0.135 ± 0.020 | 0.030 ± 0.007ab |
| mg/ml | | | | | | |
| 20 | 0.344 ± 0.061 | 4.385 ± 0.516ab | 0.617 ± 0.074 | 1.472 ± 0.147 | 0.144 ± 0.130 | 0.031 ± 0.009ab |
| mg/ml | | | | | | |
| 10 | 0.342 ± 0.032 | 4.474 ± 0.353ab | 0.631 ± 0.118 | 1.467 ± 0.147 | 0.137 ± 0.006 | 0.034 ± 0.002ab |
| mg/ml | | | | | | |

3.3.4. Hematological Change

As a result of 14 hematological tests, increases in the percentages of WBC, lymphocytes, and monocytes and a decrease in the percentage of neutrophilic leukocytes related to the increases in the percentages of WBC, lymphocytes, and monocytes were acknowledged along with significant (p<0.01) decreases in RBC, HGB, and HCT, as compared to the vehicle control, but in the groups to which the complex compositions of 16 mg/ml of gefitinib and 40, 20, or 10 mg/ml of LF were administered, decreases in the percentages of WBC, lymphocytes, and monocytes and an increase in the percentage of neutrophilic leukocytes related to the decreases in the percentages of WBC, lymphocytes, and monocytes were acknowledged along with significant (p<0.01) increases in RBC, HGB, and HCT, respectively, as compared to the group to which the single composition of 16 mg/ml of gefitinib was administered. Meanwhile, in the group to which the single composition of 40 mg/ml of LF was administered, a significant hematological change was not acknowledged as compared to the vehicle control, and even in all the groups to which the single composition of 16 mg/ml of gefitinib was administered and the complex compositions of the three types of gefitinib and LF were administered, no significant changes in MCV, MCH, MCHC, PLT, RET, EOS %, and BAS % were acknowledged, respectively, as compared to the vehicle control (Table 25).

TABLE 25

| Items | Hematological Items: Red Blood Cells | | | | | | | |
|---|---|---|---|---|---|---|---|---|
| Groups | RBC | HGB | HCT | MCV | MCH | MCHC | PLT | RET |
| Vehicle | 8.35 ± 0.54 | 16.41 ± 0.49 | 44.00 ± 1.38 | 52.94 ± 3.27 | 21.76 ± .40 | 41.69 ± 1.11 | 355.29 ± 125.82 | 0.80 ± 0.16 |
| control | | | | | | | | |

Gefitinib single formula

| | | | | | | | | |
|---|---|---|---|---|---|---|---|---|
| 16 | 6.30 ± 0.35c | 14.87 ± 0.97a | 34.06 ± 2.18a | 49.81 ± 3.97 | 21.01 ± 1.93 | 42.31 ± 4.13 | 760.00 ± 143.30 | 0.43 ± 0.37 |
| mg/ml | | | | | | | | |

LF single formula

| | | | | | | | | |
|---|---|---|---|---|---|---|---|---|
| 40 | 8.36 ± 0.49e | 19.10 ± 0.46b | 44.24 ± 1.14b | 53.04 ± 3.71 | 21.68 ± 1.36 | 40.90 ± 1.69 | 530.14 ± 182.11 | 0.37 ± 0.36 |
| mg/ml | | | | | | | | |

Gefitinib 16 mg/ml and LF mixed formula

| | | | | | | | | |
|---|---|---|---|---|---|---|---|---|
| 40 | 7.74 ± 0.42e | 17.17 ± 0.52ab | 40.97 ± 2.01ab | 53.01 ± 3.95 | 22.20 ± 1.46 | 41.91 ± 1.74 | 779.56 ± 164.72 | 0.87 ± 0.28 |
| mg/ml | | | | | | | | |
| 20 | 7.39 ± 0.26ce | 18.99 ± 0.78ab | 40.50 ± 2.35ab | 51.43 ± 3.21 | 22.67 ± 1.02 | 41.93 ± 1.17 | 815.86 ± 141.90 | 0.34 ± 0.16 |
| mg/ml | | | | | | | | |
| 10 | 7.26 ± 0.06ce | 16.20 ± 0.52ab | 36.46 ± 1.43ab | 52.90 ± 2.34 | 22.26 ± 9.76 | 42.14 ± 2.00 | 795.00 ± 117.34 | 0.29 ± 0.18 |
| mg/ml | | | | | | | | |

| | Hematological Items: White Blood Cells | | | | | |
|---|---|---|---|---|---|---|
| Items | | NEU | LYM | MONO | EOS | BASO |
| Groups | WBC | (%) | (%) | (%) | (%) | (%) |
| Vehicle | 3.67 ± 0.72 | 9.60 ± 1.35 | 35.99 ± 2.20 | 1.20 ± 0.72 | 0.44 ± 0.37 | 0.97 ± 0.46 |
| control | | | | | | |

Gefitinib single formula

| | | | | | | |
|---|---|---|---|---|---|---|
| 16 | 7.50 ± 0.49e | 2.95 ± 0.87c | 90.22 ± 0.91c | 3.59 ± 0.83a | 0.36 ± 0.24 | 1.11 ± 0.21 |
| mg/ml | | | | | | |

LF single formula

| | | | | | | |
|---|---|---|---|---|---|---|
| 40 | 3.69 ± 0.64b | 9.61 ± 1.84e | 96.27 ± 1.77e | 1.29 ± 0.50b | 0.43 ± 0.26 | 1.03 ± 0.38 |
| mg/ml | | | | | | |

Gefitinib 16 mg/ml and LF mixed formula

| | | | | | | |
|---|---|---|---|---|---|---|
| 40 | 4.65 ± 0.54ab | 7.30 ± 0.79ce | 85.41 ± 1.53de | 7.07 ± 0.47ab | 0.36 ± 0.24 | 0.96 ± 0.70 |
| mg/ml | | | | | | |
| 20 | 5.36 ± 0.57ab | 6.37 ± 0.56ce | 88.71 ± 0.80de | 2.47 ± 0.40ab | 0.43 ± 9.14 | 0.99 ± 0.49 |
| mg/ml | | | | | | |
| 10 | 5.82 ± 0.86ab | 5.79 ± 0.51ce | 88.69 ± 0.39ce | 3.52 ± 0.49ab | 0.40 ± 0.24 | 1.06 ± 0.25 |
| mg/ml | | | | | | |

3.3.5. Serum Biochemical Change

As a result of carrying out 20 serum biochemical tests, in the group to which the single composition of 16 mg/ml of gefitinib was administered, decreases in albumin and A/G were acknowledged along with significant (p<0.01) increases in contents of AST, ALT, globulin, and LDH, as compared to the vehicle control, but in the groups to which the complex compositions of 16 mg/ml of gefitinib and 10, 20, or 40 mg/ml of LF were administered, significant (p<0.01) decreases in contents of AST, ALT, globulin, and LDH and increases in albumin and A/G were acknowledged in a manner which is concentration-dependent on LF constituting the complex, respectively, as compared to the group to which the single composition of 16 mg/ml of gefitinib was administered. Meanwhile, in the group to which the single composition of 40 mg/ml of LF was administered, a significant serum biochemical change was not acknowledged as compared to the vehicle control, and in all the groups to which the single composition of 16 mg/ml of gefitinib was administered and the complex compositions of gefitinib and the three doses of LF were administered, significant changes in ALP, BUN, CRE, GLU, CHO, PRO, CPK, T-BIL, TG, Ca, P, Na, K, and Cl were not acknowledged, respectively, as compared to the vehicle control (Table 26).

TABLE 26

| Items | Serum Biochemical Items | | | | | | |
|---|---|---|---|---|---|---|---|
| Groups | AST | ALT | ALP | BUN | CRE | GLU | CHO |
| Vehicle | 72.29 ± 11.59 | 31.14 ± 7.06 | 104.86 ± 17.93 | 18.39 ± 0.79 | 0.317 ± 0.15 | 94.43 ± 5.19 | 141.57 ± 24.10 |
| control | | | | | | | |

Gefitinib single formula

| | | | | | | | |
|---|---|---|---|---|---|---|---|
| 16 | 29.014 ± 23.57d | 215.00 ± 33.87d | 107.00 ± 22.41 | 18.19 ± 0.89 | 0.39 ± 0.18 | 92.43 ± 7.93 | 142.00 ± 21.53 |
| mg/ml | | | | | | | |

LF single formula

| | | | | | | | |
|---|---|---|---|---|---|---|---|
| 40 | 72.86 ± 9.75f | 31.57 ± 2.99f | 104.71 ± 16.54 | 18.43 ± 0.71 | 0.44 ± 0.15 | 96.57 ± 10.23 | 138.86 ± 29.10 |
| mg/ml | | | | | | | |

Gefitinib 16 mg/ml and LF mixed formula

| 40 | 168.14 ± 29.11df | 109.57 ± 21.68df | 107.86 ± 17.73 | 18.41 ± 1.06 | 0.40 ± 0.16 | 91.57 ± 11.10 | 134.43 ± 31.91 |
|---|---|---|---|---|---|---|---|
| mg/ml | | | | | | | |
| 20 | 206.00 ± 26.00df | 146.57 ± 30.76df | 105.57 ± 14.26 | 18.30 ± 1.25 | 0.41 ± 0.20 | 96.43 ± 10.37 | 142.14 ± 27.72 |
| mg/ml | | | | | | | |
| 10 | 234.71 ± 29.49df | 171.86 ± 10.54df | 112.71 ± 26.09 | 16.14 ± 0.69 | 0.40 ± 0.14 | 93.43 ± 12.34 | 145.43 ± 28.42 |
| mg/ml | | | | | | | |
| Groups | | | | | | | |
| Items | PRO | CPK | BIL | ALB | Globulin | A/G | TG |
| Vehicle | 5.09 ± 0.20 | 194.57 ± 37.07 | 0.13 ± 0.05 | 2.90 ± 0.40 | 2.19 ± 0.38 | 1.40 ± 0.52 | 201.57 ± 47.40 |
| control | | | | | | | |

Gefitinib single formula

| | | | | | | | |
|---|---|---|---|---|---|---|---|
| 16 | 5.11 ± 0.19 | 192.57 ± 36.57 | 0.14 ± 0.05 | 1.39 ± 0.25a | 3.73 ± 0.39a | 0.38 ± 0.12d | 195.71 ± 47.00 |
| mg/ml | | | | | | | |

LF single formula

| | | | | | | | |
|---|---|---|---|---|---|---|---|
| 40 | 5.03 ± 0.19 | 195.00 ± 35.59 | 0.13 ± 0.08 | 2.87 ± 0.26c | 2.16 ± 0.40c | 1.36 ± 0.34f | 193.71 ± 41.25 |
| mg/ml | | | | | | | |

Gefitinib 16 mg/ml and LF mixed formula

| | | | | | | | |
|---|---|---|---|---|---|---|---|
| 40 | 5.10 ± 0.32 | 192.86 ± 28.54 | 0.13 ± 0.05 | 2.44 ± 0.26ac | 2.66 ± 0.33bc | 0.94 ± 0.17ef | 187.14 ± 20.96 |
| mg/ml | | | | | | | |
| 20 | 5.13 ± 0.21 | 190.86 ± 39.36 | 0.13 ± 0.05 | 2.16 ± 0.24ac | 2.97 ± 0.36ac | 0.74 ± 0.18df | 190.57 ± 31.25 |
| mg/ml | | | | | | | |
| 10 | 5.10 ± 0.23 | 192.86 ± 26.15 | 0.16 ± 0.08 | 1.96 ± 0.16ac | 3.14 ± 0.36ac | 0.63 ± 0.11df | 194.71 ± 29.36 |
| mg/ml | | | | | | | |

| Groups | | | | | | |
|---|---|---|---|---|---|---|
| Items | LDH (×102) | Ca | P | Na | K | Cl |
| Vehicle | 9.56 ± 1.67 | 10.43 ± 0.29 | 10.17 ± 0.19 | 141.43 ± 1.13 | 9.94 ± 0.24 | 114.43 ± 1.27 |
| control | | | | | | |

Gefitinib single formula

| | | | | | | |
|---|---|---|---|---|---|---|
| 16 | 40.62 ± 8.26d | 10.40 ± 0.28 | 10.13 ± 0.29 | 141.29 ± 1.11 | 9.97 ± 0.25 | 114.71 ± 1.60 |
| mg/ml | | | | | | |

LF single formula

| | | | | | | |
|---|---|---|---|---|---|---|
| 40 | 9.32 ± 1.28f | 10.40 ± 0.25 | 10.11 ± 0.30 | 141.57 ± 1.51 | 10.03 ± 0.21 | 114.57 ± 2.30 |
| mg/ml | | | | | | |

Gefitinib 16 mg/ml and LF mixed formula

| | | | | | | |
|---|---|---|---|---|---|---|
| 40 | 18.57 ± 6.12df | 10.49 ± 0.23 | 10.20 ± 0.26 | 141.86 ± 1.77 | 9.91 ± 0.20 | 114.43 ± 0.98 |
| mg/ml | | | | | | |
| 20 | 26.75 ± 3.98df | 10.44 ± 0.24 | 10.14 ± 0.29 | 141.43 ± 1.13 | 9.99 ± 0.42 | 114.29 ± 1.11 |
| mg/ml | | | | | | |
| 10 | 30.27 ± 1.58df | 10.41 ± 0.20 | 10.14 ± 0.22 | 141.29 ± 1.89 | 9.96 ± 0.37 | 114.86 ± 1.07 |
| mg/ml | | | | | | |

3.3.6. Autopsy Findings

In the group to which the single composition of 16 mg/ml of gefitinib was administered, increases in frequency of observing a finding of a significant discoloration of the liver and a finding of spleen and submandibular lymph node hypertrophy were acknowledged, respectively, as compared to the vehicle control, but in all the groups to which the complex compositions were administered, decreases in frequency of observing a finding of a remarkable discoloration of the liver and a finding of spleen and submandibular lymph node hypertrophy were acknowledged, respectively, as compared to the group to which the single composition of gefitinib was administered. Meanwhile, a finding of mild [1+] pulmonary hyperemia was sporadically observed in all the experimental groups including the vehicle control (Table 27).

TABLE 27

| | | Gefitinib | LF | Gefitinib 16 mg/ml | | |
|---|---|---|---|---|---|---|
| Groups | | Single | Single | and LF mixed formula | | |
| Organs/ | Vehicle | 16 | 40 | 40 | 20 | 10 |
| Findings | control | mg/ml | mg/ml | mg/ml | mg/ml | mg/ml |
| Lung | | | | | | |
| Normal | 5/7 | 5/7 | 5/7 | 6/7 | 5/7 | 6/7 |
| Congestion | 2/7 | 2/7 | 2/7 | 1/7 | 2/7 | 1/7 |
| 1+ | 2/7 | 2/7 | 2/7 | 1/7 | 2/7 | 1/7 |
| Spleen | | | | | | |
| Normal | 5/7 | 0/7 | 5/7 | 3/7 | 2/7 | 2/7 |
| Hypertrophy | 2/7 | 7/7 | 2/7 | 4/7 | 5/7 | 5/7 |
| 1+ | 2/7 | 0/7 | 2/7 | 4/7 | 2/7 | 3/7 |
| 2+ | 0/7 | 4/7 | 0/7 | 0/7 | 3/7 | 2/7 |
| 3+ | 0/7 | 3/7 | 0/7 | 0/7 | 0/7 | 0/7 |
| Liver | | | | | | |
| Normal | 7/7 | 0/7 | 7/7 | 0/7 | 0/7 | 0/7 |
| Discolorization | 0/7 | 7/7 | 0/7 | 7/7 | 7/7 | 7/7 |
| 1+ | 0/7 | 0/7 | 0/7 | 5/7 | 3/7 | 3/7 |
| 2+ | 0/7 | 1/7 | 0/7 | 2/7 | 4/7 | 3/7 |
| 3+ | 0/7 | 6/7 | 0/7 | 0/7 | 0/7 | 1/7 |
| Lymph nodes) | | | | | | |
| Normal | 6/7 | 0/7 | 7/7 | 2/7 | 2/7 | 1/7 |
| Hypertrophy | 1/7 | 7/7 | 0/7 | 5/7 | 5/7 | 6/7 |
| 1+ | 1/7 | 0/7 | 0/7 | 3/7 | 4/7 | 5/7 |
| 2+ | 0/7 | 4/7 | 0/7 | 2/7 | 1/7 | 1/7 |
| 3+ | 0/7 | 3/7 | 0/7 | 0/7 | 0/7 | 0/7 |
| Others | | | | | | |
| Normal | 7/7 | 7/7 | 7/7 | 7/7 | 7/7 | 7/7 |

3.3.7. Histopathological Observation

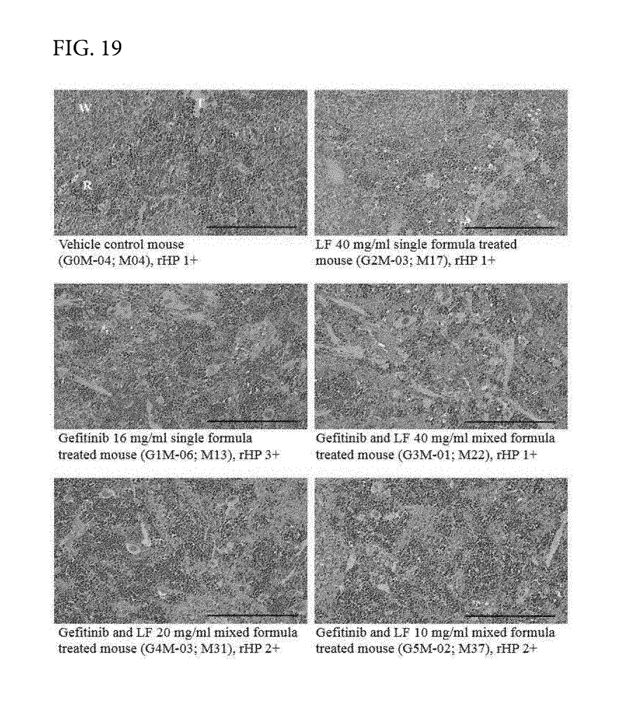

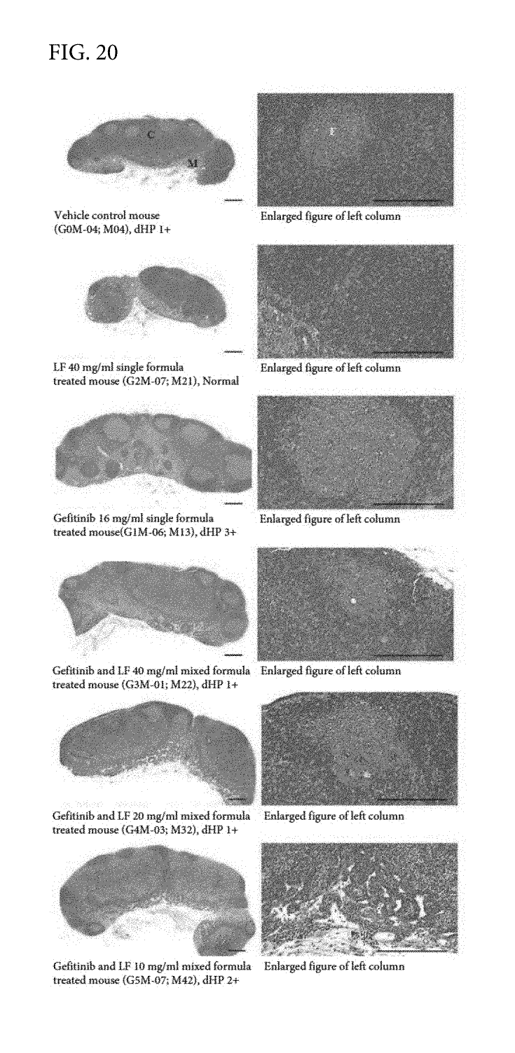

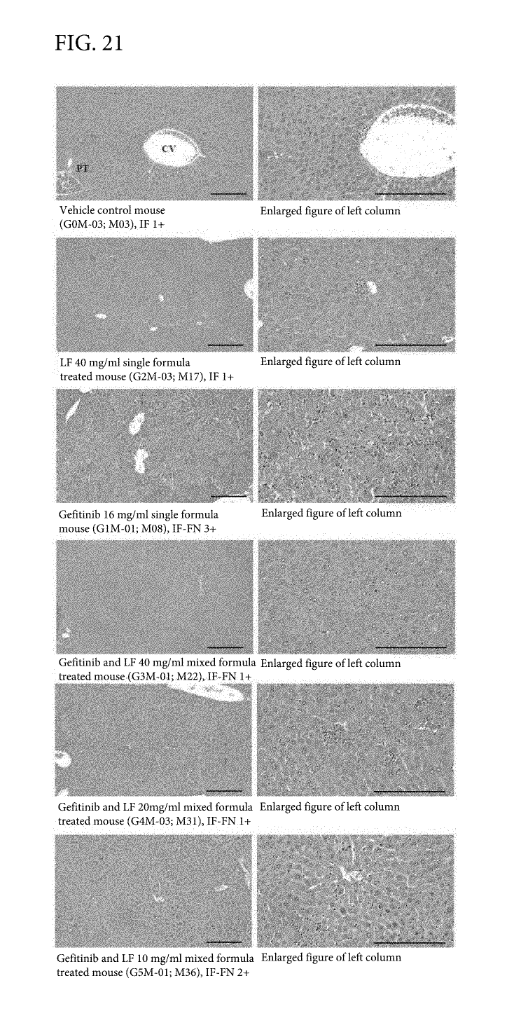

In the vehicle control, findings of mild hyperplasia of splenic red pulp lymphocytes (FIG. 19), which was limited to the first case (1/7; 14.29%), diffused hyperplasia of submandibular lymph node lymphocytes (FIG. 20), and infiltration of local inflammatory cells into the liver (FIG. 21) were acknowledged, and in the group to which the single composition of 40 mg/ml of LF was administered, findings of mild infiltration of local inflammatory cells into the liver (FIG. 21), which was limited to the first case (1/7; 14.29%) and mild hyperplasia of splenic red pulp lymphocytes (FIG. 21) in the second case (28.57%) were acknowledged, but in the group to which the single composition of 16 mg/ml of gefitinib was administered, an increase in frequency of findings of and observations of various degrees (mild, moderate [2+] or severe [3+]) of local necrosis of the liver and infiltration of inflammatory cells (FIG. 21), diffused hyperplasia of submandibular lymph node lymphocytes (FIG. 20), and hyperplasia of splenic red pulp lymphocytes (FIG. 19) were acknowledged, respectively, as compared to the vehicle control. Meanwhile, in the groups to which the complex compositions of gefitinib and 10, 20, or 40 mg/ml of LF were administered, remarkable decreases in frequency of findings of hyperplasia of splenic red pulp and submandibular lymph node lymphocytes and findings of and the appearance of local necrosis of the liver accompanied by infiltration of inflammatory cells were acknowledged, respectively, as compared to the group to which the single composition of 16 mg/ml of gefitinib was administered.

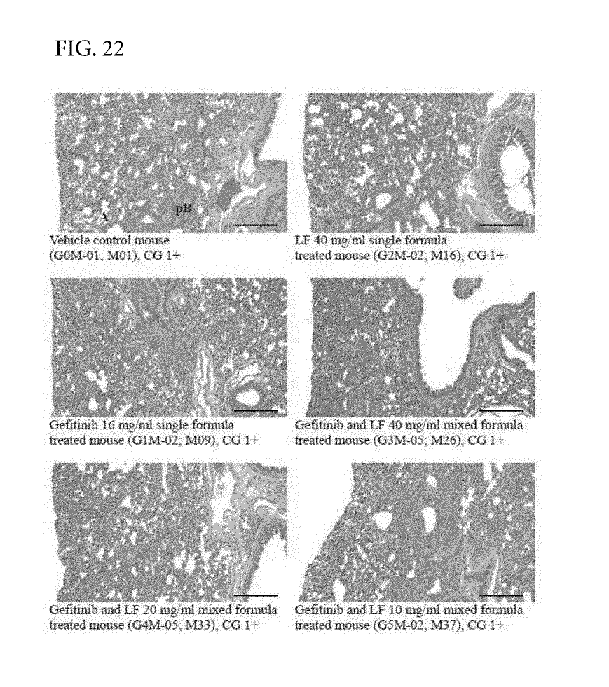

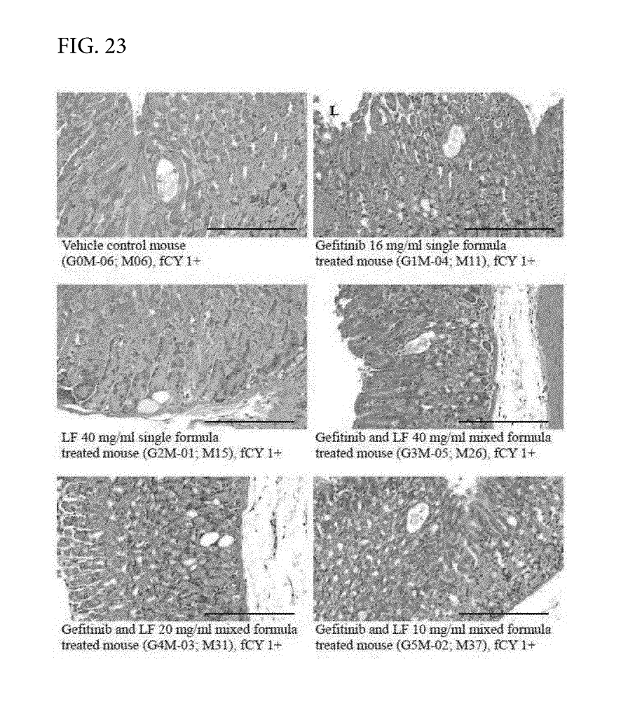

Meanwhile, a finding of mild pulmonary hyperemia (FIG. 22) or formation of local cysts of the cardiac stomach (FIG. 23) was sporadically observed in all the experimental groups including the vehicle control.

TABLE 28

| | | Gefitinib | LF | Gefitinib 16 mg/ml | | |
|---|---|---|---|---|---|---|
| | | Single | Single | and LF mixed formula | | |
| | Vehicle | 16 | 40 | 40 | 20 | 10 |
| Groups | control | mg/ml | mg/ml | mg/ml | mg/ml | mg/ml |
| Lung | | | | | | |
| Normal | 5/7 | 6/7 | 5/7 | 6/7 | 5/7 | 6/7 |
| CG† 1+ | 2/7 | 1/7 | 2/7 | 1/7 | 2/7 | 1/7 |
| Spleen | | | | | | |
| Normal | 6/7 | 0/7 | 5/7 | 4/7 | 2/7 | 2/7 |
| rHP†1+ | 1/7 | 0/7 | 2/7 | 3/7 | 2/7 | 2/7 |
| 2+ | 0/7 | 3/7 | 0/7 | 0/7 | 3/7 | 3/7 |
| 3+ | 0/7 | 4/7 | 0/7 | 0/7 | 0/7 | 0/7 |
| Liver | | | | | | |
| Normal | 6/7 | 0/7 | 6/7 | 0/7 | 0/7 | 0/7 |
| IF† 1+ | 1/7 | 0/7 | 1/7 | 0/7 | 0/7 | 0/7 |
| IF-FN†1+ | 0/7 | 0/7 | 0/7 | 5/7 | 3/7 | 3/7 |
| 2+ | 0/7 | 1/7 | 0/7 | 2/7 | 4/7 | 4/7 |
| 3+ | 0/7 | 6/7 | 0/7 | 0/7 | 0/7 | 0/7 |
| Lymph nodes) | | | | | | |
| Normal | 6/7 | 0/7 | 7/7 | 2/7 | 2/7 | 1/7 |
| dHP† 1+ | 1/7 | 0/7 | 0/7 | 3/7 | 4/7 | 5/7 |
| 2+ | 0/7 | 4/7 | 0/7 | 2/7 | 1/7 | 1/7 |
| 3+ | 0/7 | 3/7 | 0/7 | 0/7 | 0/7 | 0/7 |
| Fundus | | | | | | |
| Normal | 4/7 | 5/7 | 6/7 | 5/7 | 4/7 | 5/7 |
| fCV†1+ | 3/7 | 2/7 | 1/7 | 2/7 | 3/7 | 2/7 |
| Others | | | | | | |
| Normal | 7/7 | 7/7 | 7/7 | 7/7 | 7/7 | 7/7 |

In the group to which the single composition of 16 mg/ml of gefitinib was administered, a significant (p<0.01) increase in liver lipid peroxidation and significant (p<0.01) decreases in content of an endogenous antioxidant GSH, and endogenous antioxidant enzyme, SOD and CAT, activities were acknowledged, respectively, as compared to the vehicle control, but in the groups to which the complex compositions of gefitinib and 10, 20, or 40 mg/ml of LF were administered, a significant (p<0.01) suppression of liver lipid peroxidation and a significant increase in content of GSH or SOD and CAT activities were acknowledged in a manner which is concentration-dependent on LF in the complex composition, respectively, as compared to the group to which the single composition of 16 mg/ml of gefitinib was administered. Meanwhile, in the group to which the single composition of 40 mg/ml of LF was administered, significant changes in liver lipid peroxidation and antioxidant defense systems were not acknowledged as compared to the vehicle control (Table 29).

TABLE 29

| Items | LPO | Antioxidative Defense Systems | | |
|---|---|---|---|---|
| (Unit) | MDA levels | Glutathione | CAT | |
| Groups | (nM/mg tissue) | (μM/mg tissue) | (U/mg tissue) | SOD (U/mg tissue) |

Vehicle control

| | | | | |
|---|---|---|---|---|
| Distilled water | 18.22 ± 3.12 | 37.27 ± 10.04 | 20.17 ± 2.19 | 3.01 ± 0.61 |

Gefitinib single formula

| | | | | |
|---|---|---|---|---|
| 16 mg/ml | 41.85 ± 4.01a | 11.60 ± 2.71c | 8.60 ± 3.28c | 0.57 ± 0.21c |

LF single formula

| | | | | |
|---|---|---|---|---|
| 40 mg/ml | 18.65 ± 3.03b | 40.50 ± 11.03e | 20.87 ± 3.64e | 3.31 ± 0.83e |

Gefitinib 16 mg/ml and LF mixed formula

| | | | | |
|---|---|---|---|---|
| 40 mg/ml | 24.23 ± 4.28ab | 27.22 ± 4.32e | 17.54 ± 1.40de | 2.28 ± 0.38de |
| 20 mg/ml | 31.30 ± 2.32ab | 24.53 ± 4.75de | 15.52 ± 2.06ce | 1.59 ± 0.49ce |
| 10 mg/ml | 33.99 ± 3.51ab | 20.08 ± 3.00ce | 13.81 ± 1.19ce | 1.12 ± 0.23ce |

For the liver lipid peroxidation, the group to which the single composition of 16 mg/ml of gefitinib was administered exhibited a change of 129.64% as compared to the vehicle control, and the group to which the single composition of 40 mg/ml of LF was administered and the groups to which the complex compositions of gefitinib and 10, 20, or 40 mg/ml of LF were administered exhibited a change of −55.43, −42.11, −25.21, and −18.77%, respectively, as compared to the group to which the single composition of 16 mg/ml of gefitinib was administered.

For the content of GSH in the liver tissue, the group to which the single composition of 16 mg/ml of gefitinib was administered exhibited a change of −69.14% as compared to the vehicle control, and the group to which the single composition of 40 mg/ml of LF was administered and the groups to which the complex compositions of gefitinib and 10, 20, or 40 mg/ml of LF were administered exhibited a change of 252.20, 136.71, 113.27, and 74.60%, respectively, as compared to the group to which the single composition of 16 mg/ml of gefitinib was administered.

For the CAT activity in the liver tissue, the group to which the single composition of 16 mg/ml of gefitinib was administered exhibited a change of −57.37% as compared to the vehicle control, and the group to which the single composition of 40 mg/ml of LF was administered and the groups to which the complex compositions of gefitinib and 10, 20, or 40 mg/ml of LF were administered exhibited a change of 142.66, 103.99, 80.47, and 60.53%, respectively, as compared to the group to which the single composition of 16 mg/ml of gefitinib was administered.

For the SOD activity in the liver tissue, the group to which the single composition of 16 mg/ml of gefitinib was administered exhibited a change of −81.18% as compared to the vehicle control, and the group to which the single composition of 40 mg/ml of LF was administered and the groups to which the complex compositions of gefitinib and 10, 20, or 40 mg/ml of LF were administered exhibited a change of 485.35, 303.28, 180.81, and 97.47%, respectively, as compared to the group to which the single composition of 16 mg/ml of gefitinib was administered.

As can be confirmed from the results in the present Example 3, cases of death and clinical symptoms related to the administration of a single composition of 16 mg/ml of gefitinib or 40 mg/ml of LF were not acknowledged throughout the entire experimental period, but in the group to which the single composition of 16 mg/ml of gefitinib was administered, an increase in weight of the spleen, liver, and submandibular lymph node, decreases in RBC, HGB, and HCT, increases in the percentages of WBC, lymphocytes, and monocytes, and a decrease in the percentage of neutrophilic leukocytes related to the increases in the percentages of WBC, lymphocytes, and monocytes, increases in serum AST, ALT, LDH, and globulin contents, and decreases in ALB and A/G were acknowledged, and in particular, in gross and histopathological examinations, findings of remarkable hyperplasia of splenic red pulp and submandibular lymph node lymphocytes, spleen and submandibular lymph node hypertrophy due to a finding of local necrosis accompanied by infiltration of inflammatory cells into the liver, and a finding of discoloration of the liver were acknowledged, respectively. In addition, an increase in liver lipid peroxidation and a decrease in content or activity of endogenous antioxidant and related enzymes (GSH, SOD, and CAT) were acknowledged, respectively. Meanwhile, a finding of anemia and hepatotoxicity caused by the gefitinib and a finding of secondary spleen and lymph node hypertrophy were remarkably suppressed in a manner which is concentration-dependent on LF constituting the complex, and a significant decrease in liver lipid peroxidation and a significant increase in activity of the antioxidant defense system were acknowledged, respectively, as compared to the group to which the single composition of 16 mg/ml of gefitinib was administered. Accordingly, it is determined that as in Examples 1 and 2, the administration of the complex composition of gefitinib and LF remarkably decreases the general toxic symptoms (anemia and hepatotoxicity) of gefitinib through the immune regulation and antioxidant effects which are already well known without affecting the bioavailability of gefitinib, so that it is expected that the complex composition of gefitinib and LF can provide a therapeutic method which is very useful for cooperative diagnosis by Oriental and Western medicine and treatment of patients with lung cancer. Meanwhile, in the group to which the single composition of 40 mg/ml of LF was administered, significant clinical symptoms, hematological, serum biochemical, gross autopsy, and histopathological changes were not acknowledged as compared to the vehicle control.

The mice in the vehicle group used in the present Example 3 exhibited an increase in body weight within a range of increase in body weight of normal mice of the same age, but in the group to which the single composition of 16 mg/ml of gefitinib was administered, a significant decrease in body weight gain throughout the entire administration period (Day 0 to 28) was acknowledged as compared to the vehicle control, the group to which the complex composition of 16 mg/ml of gefitinib and 20 mg/ml of LF was administered exhibited a significant decrease in body weight gain for Day 0 to 14 as compared to the vehicle control, and the group to which the complex composition of 16 mg/ml of gefitinib and 100 mg/ml of LF was administered exhibited significant decreases in body weight for Day 14 to 27 and Day 0 to 28, respectively as compared to the vehicle control. The decrease in body weight gain is thought to be due to the toxicity of gefitinib itself. Meanwhile, in all the groups to which the complex compositions were administered, significant changes in body weight and body weight gains were not acknowledged as compared to the group to which the single composition of 16 mg/ml of gefitinib was administered, and even in the groups to which the single composition of 40 mg/ml of LF was administered and the complex composition of 16 mg/ml of gefitinib and 40 mg/ml of LF was administered, significant changes in body weight and body weight gain were not acknowledged throughout the entire experimental period as compared to the vehicle control. It is determined that these results are direct evidence that the complex composition of LF does not particularly affect a decrease in body weight gain caused by gefitinib at least under the conditions in the present Example.

In the present Example 3, the finding of discoloration of the liver acknowledged in the group to which the single composition of 16 mg/ml of gefitinib was administered was observed to be due to local necrosis accompanied by infiltration of inflammatory cells into the liver as a result of a histopathological examination, and an increase in liver weight caused by the discoloration of the liver was also acknowledged. Further, a decrease in content of GSH which is an endogenous antioxidant along with an increase in liver lipid peroxidation which is harmful, and a decrease in activity of SOD and CAT which are antioxidant enzymes were also acknowledged, and increases in serum AST, ALT, and LDH contents, which are blood chemical numerical values indicating damage to the liver, and a decrease in content of albumin were acknowledged. Accordingly, it is determined that the oral administration of gefitinib also caused the hepatotoxicity caused by damage to the antioxidant defense system of the liver in the present Example 3. Further, it is determined that an increase in content of serum globulin content, a decrease in A/G related to the decrease in serum globulin content, hyperplasia of spleen and submandibular lymph node lymphocytes, a finding of spleen and submandibular lymph node hypertrophy related to the hyperplasia, an increase in weight thereof, increases in the percentages of serum WBC, lymphocytes, and monocytes, a decrease in the percentage of neutrophilic leukocytes related to the increases in the percentages of WBC, lymphocytes, and monocytes, and the like are typical findings in chronic inflammation, and are secondary changes caused by a chronic inflammatory response due to damage to the liver, which results from the administration of gefitinib. Meanwhile, the finding of damage to the liver by the gefitinib and changes in the lymphatic system, spleen, and submandibular lymph node related to the damage to the liver were remarkably suppressed in a manner which is concentration-dependent on LF constituting the complex, and in particular, in the groups to the complex compositions of 16 mg/ml of gefitinib and 40, 20, and 10 mg/ml of LF were administered, a significant decrease in liver lipid peroxidation, a significant increase in content of GSH, and a significant increase in activity of SOD and CAT were acknowledged in a manner which is concentration-dependent on LF, respectively, as compared to the group to which the single composition of 16 mg/ml of gefitinib was administered. Accordingly, it is determined that the complex composition of LF at a concentration of 10 mg/ml or more remarkably suppresses gefitinib from damaging the liver through activation of the antioxidant defense system.

In a hematological examination, decreases in RBC, HGB, and HCT indicate anemia, and the possibility of causing anemia by the administration of gefitinib is already well known. Even in the present Example 3, in the group to which the single composition of 16 mg/ml of gefitinib was administered, remarkable decreases in RBC, HGB, and HCT were acknowledged, but in all the groups to which the complex compositions of the three types of gefitinib and LF, remarkable increases in RBC, HGB, and HCT were acknowledged in a manner which is dose-dependent on LF, respectively, as compared to the group to which the single composition of 16 mg/ml of gefitinib was administered, so that it is determined that a complex composition of LF at 10 mg/ml or more also remarkably suppresses the finding of anemia. Meanwhile, in all the groups to which the experimental materials were administered, significant changes in ALP, BUN, CRE, GLU, CHO, PRO, CPK, T-BIL, TG, Ca, P, Na, K, and Cl were not acknowledged as compared to the vehicle control. The finding of mild pulmonary hyperemia acknowledged during the gross autopsy, the finding of mild pulmonary hyperemia or formation of local cysts of the cardiac stomach observed during the histopathological examination, and the like are sporadically observed in all the experimental groups including the vehicle control, and thus are thought to be accidental lesions rather than toxic symptoms resulting from the administration of the experimental materials, and these symptoms are findings rarely acknowledged even in normal mice.

As described above, it was observed that the complex composition of 40, 20, or 10 mg/ml of LF remarkably suppresses damage to the liver due to anemia and the failure of the antioxidant system caused by gefitinib through immune regulation and antioxidant effects of LF itself. Accordingly, it is determined that anemia and damage to the liver caused by gefitinib are remarkably decreased through the immune activity and the activity of the antioxidant defense system, so that it is expected that it is possible to provide a new means for cooperative diagnosis by Oriental and Western medicine and treatment, which is very useful for patients with lung cancer.

A composition for the treatment or prevention of lung cancer, containing the Lonicera japonica extract provided by the present invention as an active ingredient is administered in combination with gefitinib, and has effects of increasing the efficiency of treatment of lung cancer and simultaneously reducing side effects at the time of administering gefitinib alone.

The above-described description of the present invention is provided for illustrative purposes, and the person skilled in the art to which the present invention pertains will understand that the present invention can be easily modified into other specific forms without changing the technical spirit or essential features of the present invention. Therefore, it should be understood that the above-described embodiments are illustrative only in all aspects and are not restrictive.

1. A method for treatment or prevention of lung cancer comprising:
administering a composition comprising gefitinib, Lonicera japonica extract or a mixture thereof as an active ingredient to a subject in need thereof.

2. The method of claim 1, wherein the composition reduces side effects caused by treatment of lung cancer.

3. The method of claim 1, wherein the gefitinib and the Lonicera japonica extract are formulated by being mixed in advance, or are formulated separately.

4. The method of claim 1, wherein the gefitinib and the Lonicera japonica extract are administered parenterally, orally, locoregionally, or percutaneously.

5. The method of claim 1, wherein the Lonicera japonica extract is administered between 5 minutes and 4 hours after the administration of the gefitinib.
